(19) United States
(12) Patent Application Publication
(10) Pub. No.: US 2008/0020003 A1
(43) Pub. Date: Jan. 24, 2008

(54) Methods for the delivery of a beta2 agonist to induce bronchodilation and formulations for use in the same

(75) Inventors: Malcolm Hill, Solana Beach, CA (US); Hemant Deshmukh, San Diego, CA (US); Elaine Phillips, San Diego, CA (US)

Correspondence Address: WILSON SONSINI GOODRICH & ROSATI, 650 PAGE MILL ROAD, PALO ALTO, CA, 94304-1050, US

(73) Assignee: Verus Pharmaceuticals, Inc., San Diego, CA (US)

(21) Appl. No.: 11/804,724

(22) Filed: May 18, 2007

Related U.S. Application Data
(60) Provisional application No. 60/747,657, filed on May 18, 2006.
(60) Provisional application No. 60/803,232, filed on May 25, 2006.
(60) Provisional application No. 60/828,212, filed on Oct. 4, 2006.
(60) Provisional application No. 60/828,215, filed on Oct. 4, 2006.

(51) Int. Cl. A61K 31/133; A61K 9/72; A61P 11/00

(52) U.S. Cl. 424/400; 514/653

(57) ABSTRACT

The present invention relates to method for inducing bronchodilation in a patient in need thereof comprising (a) providing at least one dose of an inhalation mixture comprising a β₂ agonist to said patient; and (b) delivering said inhalation mixture with an inhalation nebulizer, as well as dosage formulations comprising a β₂ agonist.

### RELATED APPLICATIONS

This application claims the benefit of U.S. Provisional Application Nos. 60/747,657, filed May 18, 2006; 60/803,232, filed May 25, 2006; 60/828,212, filed Oct. 4, 2006; and 60/828,215, filed Oct. 4, 2006, which are hereby incorporated by reference in their entireties.

### FIELD OF THE INVENTION

The present invention relates to methods for the delivery of a 02 agonist to induce bronchodilation in a patient in need thereof comprising (a) providing at least one dose of an inhalable mixture comprising a β₂ agonist and (b) delivering the inhalation mixture with an inhalation nebulizer, as well as dosage formulations comprising a β₂ agonist.

### BACKGROUND OF THE INVENTION

Bronchoconstrictive disorders can include such pulmonary diseases as asthma and its related disorders, including pediatric asthma, bronchial asthma, allergic asthma, occupational asthma, aspirin sensitive asthma, intrinsic asthma, and chronic obstructive pulmonary disease (COPD), and chronic bronchitis. Such bronchoconstrictive disorders are widespread and affect millions of people worldwide.

The pathophysiology of many bronchoconstrictive disorders, including, asthma, involves various distinct symptoms, one of which is bronchoconstriction, which can result in wheezing, coughing and shortness of breath. It is believed that in these disorders, bronchoconstriction occurs due to one or more of bronchial smooth muscle spasms, airway inflammation and bronchial mucosal edema.

In particular, asthma is a bronchoconstrictive disorder marked by (a) labored breathing; (b) wheezing; and (c) coughing. Like many bronchoconstrictive disorders, asthma is characterized by: (1) airway inflammation; (2) airway hyper-responsiveness; and (3) airway narrowing. However, the severity of these symptoms can vary widely from patient to patient and even from one asthmatic episode (attack) to the next within the same patient.

β₂ agonists, also known in the art as β₂-adrenergic receptor agonists, are known to provide a bronchodilatory effect in humans and are important in the treatment of patients suffering from bronchoconstrictive disorders because the administration of β₂ agonists results in relief from the symptoms of breathlessness. The β₂ agonists can be short acting for immediate relief, or long acting for long-term prevention, of bronchoconstrictive symptoms. For example, known short acting β₂ agonists include albuterol, biltolterol, levalbuterol, pirbuterol, salbutamol, and terbutaline. Additionally, known long acting β₂ agonists include arformoterol, arformoterol tartrate, formoterol and salmeterol.

More specifically, short-acting inhaled β₂ agonists, such as albuterol, are used to prevent and treat wheezing, shortness of breath, and troubled breathing caused by asthma, chronic bronchitis, emphysema, and other lung diseases. β₂ agonist inhalation is also used to prevent breathing difficulties (bronchospasm) during exercise. Currently, albuterol is available as a tablet, extended-release (long-acting) tablet, and a syrup to take by mouth and as an aerosol, a solution (liquid), and a powder-filled capsule to inhale by mouth. The solution is inhaled using a nebulizer, and the powder-filled capsules are inhaled using a special dry powder inhaler. Albuterol tablets and syrup are usually taken three or four times a day, and extended-release tablets are usually taken twice a day. For the treatment or prevention of asthma symptoms, the oral inhalation is usually used every 4 to 6 hours as needed. For the prevention of bronchospasm during exercise, the oral inhalation is used 15 minutes before exercise. The nebulized solution is used three or four times a day.

The inhalation solution of albuterol is currently available in 2.5 mg, 1.25 mg, and 0.63 mg unit doses in 3 mls of an isotonic aqueous solution (Albuterol Sulfate Inhalation Solution and Accuneb®, respectively (Dey, L. P.). The 2.5 mg dose has been approved for use by adults, and the FDA has likewise expanded labeling guidelines to include this amount of albuterol for use by pediatric asthmatic patients as young as 2 years old. Although, when administered on a regular basis to a child, the 2.5 mg formulation may provide more albuterol than needed, and thereby increase the risk of adverse drug side effects. As such, the National Institutes of Health (NIH) has recommended that pediatric patients use the lowest β₂ agonist dose needed to control symptoms.

Thus, irrespective of the dosage form used for administration of β₂ agonists, current methods of treatment using β₂ agonists require patients to comply with different dosage regimens, different frequencies of administration and substantial difficulties in patient compliance. Further, certain patient populations, e.g., pediatric asthmatic patients, present particular difficulties with regard to β₂ agonist nebulization therapies due the unwillingness of that patient population to comply with the required length of time necessary to administer traditional β₂ agonist nebulization therapies.

Accordingly, there is a need for improved methods, and dosage formulations for use therein, for the delivery of inhalation mixtures comprising β₂ agonists to induce bronchodilation in a patient in need thereof, wherein the β₂ agonists are delivered in a manner in which β₂ agonist dosage is lowered, wherein administration time is reduced, and wherein the risk of side effects related to β₂ agonist therapy is diminished.

The present invention meets the foregoing needs and provides related advantages as well.

### SUMMARY OF THE INVENTION

The present invention meets the foregoing and related needs by providing an improved method of treating bronchoconstrictive disorders, including asthma, with β₂ agonists where current treatments are not ideal.

In one embodiment of the present invention, the invention comprises a method for treating a disease by inducing bronchodilation in a patient in need thereof comprising: (a) providing at least one dose of an inhalation mixture comprising a β₂ agonist to said patient; and (b) delivering said inhalation mixture with an inhalation nebulizer, wherein the β₂ agonist is provided at a concentration of less than about 0.21 mg/dose and whereby the delivering with said inhalation nebulizer is for less than about 5 minutes. In other embodiments of the methods described herein, the β₂ agonist is administered at a concentration of less than about 0.18 mg/dose. In other embodiments, the β₂ agonist is administered at a concentration of less than about 0.16 mg/dose. In still other embodiments, the β₂ agonist is administered at a concentration of less than about 0.14 mg/dose. In yet other embodiments, the β₂ agonist is administered at a concentration of less than about 0.12 mg/dose. In still yet other embodiments, the β₂ agonist is administered at a concentration of less than about 0.10 mg/dose. In one embodiment, the β₂ agonist is administered at a concentration of less than about 0.08 mg/dose. In another embodiment, the β₂ agonist is administered at a concentration of less than about 0.06 mg/dose. In yet another embodiment, the β₂ agonist is administered at a concentration of less than about 0.04 mg/dose.

In another embodiment of the present invention, the invention comprises a method for treating a disease by inducing bronchodilation in a patient in need thereof comprising: (a) providing at least one dose of an inhalation mixture comprising a β₂ agonist to said patient; and (b) delivering the inhalation mixture with an inhalation nebulizer for less than about 5 minutes, wherein at least about 20% of the β₂ agonist is deposited in the lung. In one embodiment, at least about 30% of the β₂ agonist is deposited in the lung. In another embodiment, at least about 40% of the β₂ agonist is deposited in the lung. In still another embodiment, at least about 50%, of the β₂ agonist is deposited in the lung. In yet still another embodiment, at least about 60% of the β₂ agonist is deposited in the lung. In another embodiment, at least about 70% of the β₂ agonist is deposited in the lung. In yet another embodiment, at least about 80% of the β₂ agonist is deposited in the lung. In certain embodiments of the methods described herein, the β₂ agonist is administered at a concentration of less than about 0.21 mg/dose. In other embodiments of the methods described herein, the β₂ agonist is administered at a concentration of less than about 0.18 mg/dose. In other embodiments, the β₂ agonist is administered at a concentration of less than about 0.16 mg/dose. In still other embodiments, the β₂ agonist is administered at a concentration of less than about 0.14 mg/dose. In yet other embodiments, the β₂ agonist is administered at a concentration of less than about 0.12 mg/dose. In still yet other embodiments, the β₂ agonist is administered at a concentration of less than about 0.10 mg/dose. In one embodiment, the β₂ agonist is administered at a concentration of less than about 0.08 mg/dose. In another embodiment, the β₂ agonist is administered at a concentration of less than about 0.06 mg/dose. In yet another embodiment, the β₂ agonist is administered at a concentration of less than about 0.04 mg/dose.

In yet another embodiment of the present invention, the invention comprises a method for treating a disease by inducing bronchodilation in a patient in need thereof comprising: (a) providing at least one dose of an inhalation mixture comprising a β₂ agonist to said patient; and (b) delivering the inhalation mixture with an inhalation nebulizer for less than about 5 minutes, wherein less than about 30% of the β₂ agonist is delivered outside of the lung. In one embodiment, less than about 25% of the β₂ agonist is delivered outside of the lung. In another embodiment, less than about 20% of the β₂ agonist is delivered outside of the lung. In yet another embodiment, less than about 15% of the β₂ agonist is delivered outside of the lung. In still another embodiment, less than about 10% of the β₂ agonist is delivered outside of the lung. In yet still another embodiment, less than about 5% of the β₂ agonist is delivered outside the lung. In certain embodiments of the methods described herein, the β₂ agonist is administered at a concentration of less than about 0.21 mg/dose. In other embodiments of the methods described herein, the β₂ agonist is administered at a concentration of less than about 0.18 mg/dose. In other embodiments, the β₂ agonist is administered at a concentration of less than about 0.16 mg/dose. In still other embodiments, the β₂ agonist is administered at a concentration of less than about 0.14 mg/dose. In yet other embodiments, the β₂ agonist is administered at a concentration of less than about 0.12 mg/dose. In still yet other embodiments, the β₂ agonist is administered at a concentration of less than about 0.10 mg/dose. In one embodiment, the β₂ agonist is administered at a concentration of less than about 0.08 mg/dose. In another embodiment, the β₂ agonist is administered at a concentration of less than about 0.06 mg/dose. In yet another embodiment, the β₂ agonist is administered at a concentration of less than about 0.04 mg/dose.

In certain embodiments of the present invention, the invention comprises a method for treating a disease by inducing bronchodilation in a patient in need thereof comprising: (a) providing at least one dose of an inhalation mixture comprising a β₂ agonist to said patient; and (b) delivering said inhalation mixture with an inhalation nebulizer for less than about 5 minutes, wherein the β₂ agonist is provided at a concentration of between about 0.04 mg/dose to about 0.1 mg/dose. In certain other embodiments, the β₂ agonist is administered at a concentration of between about 0.1 mg/dose to about 0.5 mg/dose. In still other embodiments, the β₂ agonist is administered at a concentration of between about 0.6 mg/dose to about 1.0 mg/dose. In yet other embodiments, the β₂ agonist is administered at a concentration of between about 1.0 mg/dose to about 1.5 mg/dose. In still yet other embodiments, the β₂ agonist is administered at a concentration of between about 1.5 mg/dose to about 2.0 mg/dose. In other embodiments of the methods described herein, the β₂ agonist is administered at a concentration of less than about 0.18 mg/dose. In other embodiments, the β₂ agonist is administered at a concentration of less than about 0.16 mg/dose. In still other embodiments, the β₂ agonist is administered at a concentration of less than about 0.14 mg/dose. In yet other embodiments, the β₂ agonist is administered at a concentration of less than about 0.12 mg/dose. In still yet other embodiments, the β₂ agonist is administered at a concentration of less than about 0.10 mg/dose. In one embodiment, the o agonist is administered at a concentration of less than about 0.08 mg/dose. In another embodiment, the β₂ agonist is administered at a concentration of less than about 0.06 mg/dose. In yet another embodiment, the β₂ agonist is administered at a concentration of less than about 0.04 mg/dose.

In still another embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein at least about 25% of the β₂ agonist is deposited in the lung. In one embodiment, at least about 30% of the β₂ agonist is deposited in the lung. In another embodiment, at least about 35% of the β₂ agonist is deposited in the lung. In yet another embodiment, at least about 40% of the β₂ agonist is deposited in the lung. In still another embodiment, at least about 50% of the β₂ agonist is deposited in the lung. In another embodiment, at least about 60% of the β₂ agonist is deposited in the lung. In yet another embodiment, at least about 80% of the β₂ agonist is deposited in the lung. In still yet another embodiment, between about 30% and about 40% of the β₂ agonist is deposited in the lung. In still yet another embodiment, between about 30% and about 60% of the β₂ agonist is deposited in the lung. In another embodiment, between about 30% and about 50% of the β₂ agonist is deposited in the lung. In yet another embodiment, between about 30% and about 40% of the β₂ agonist is deposited in the lung. In still another embodiment, between about 40% and about 50% of the β₂ agonist is deposited in the lung

In a further embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein said method reduces the risk of side effects associated with traditional β₂ agonist treatments.

In a further embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein said method reduces the side effects associated with traditional β₂ agonist treatments. In certain embodiments, the methods described herein diminish the side effects associated with traditional β₂ agonist treatments. In other embodiments, the methods described herein eliminate the side effects associated with traditional β₂ agonist treatments.

In still another embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein said method provides equivalent bronchodilation-in a patient as compared to traditional β₂ agonist treatments at a β₂ agonist dose that is lower than traditional β₂ agonist treatments.

In yet another embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, whereby said method provides greater bronchodilation in a patient as compared to traditional β2 agonist treatments wherein the β₂ agonist concentration in the present invention is the same as the β₂ agonist concentration in a traditional β₂ agonist treatment.

In certain embodiments of the present invention, the β₂ agonist is a short acting β₂ agonist selected from the group consisting of albuterol, albuterol free base, albuterol sulfate, albuterol hydrochloride, albuterol maleate, albuterol tartrate, albuterol citrate, albuterol phosphate, terbutaline sulfate, bitolterol mesylate, levalbuterol, metaproterenol sulfate, pirbuterol acetate, and combinations thereof.

In other embodiments, the short acting β₂ agonist is selected from the group consisting of albuterol, albuterol free base, albuterol sulfate, albuterol hydrochloride, albuterol maleate, albuterol tartrate, albuterol citrate, albuterol phosphate, terbutaline sulfate, levalbuterol, pirbuterol acetate, and combinations thereof.

In certain other embodiments, the short acting β₂ agonist is selected from the group consisting of albuterol, albuterol free base, albuterol sulfate, albuterol hydrochloride, albuterol citrate, albuterol phosphate, or levalbuterol.

In one embodiment, the short acting β₂ agonist is albuterol free base. In another embodiment, the short acting β₂ agonist is albuterol sulfate. In still another embodiment, the short acting β₂ agonist is albuterol hydrochloride. In still yet another embodiment, the short acting β₂ agonist is albuterol citrate. In yet still another embodiment, the short acting β₂ agonist is albuterol phosphate. In yet another embodiment, the short acting β₂ agonist is levalbuterol.

In certain other embodiments of the present invention, the β₂ agonist is a long acting β₂ agonist selected from the group consisting of formoterol, arformoterol, arformoterol tartrate, salmeterol, or combinations thereof.

In other embodiments of the present invention, the methods can further comprise the delivery of a second pharmaceutically active agent in an inhalation mixture and delivered with an inhalation nebulizer. In one embodiment, the inhalation mixture comprising a β₂ agonist and the inhalation mixture comprising a second pharmaceutically active agent are delivered at the same time, for example, in a inhalation mixture. In another embodiment, an inhalation mixture comprising a β₂ agonist and the inhalation mixture comprising a second pharmaceutically active agent are delivered consecutively.

In certain embodiments, the second pharmaceutically active agent is a corticosteroid. In other embodiments, the second pharmaceutically active agent is a corticosteroid selected from the group consisting of aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In one embodiment, the second pharmaceutically active agent is budesonide.

In other embodiments, the second pharmaceutically active agent is an antibiotic. In certain of these embodiments, the antibiotic is selected from the group consisting of penicillins, cephalosporins, macrolides, sulfonamides, aminoglycbsides, and β-lactam antibiotics.

In certain embodiments of the present invention, the patient can be an adult over 18 years of age. In other embodiments of the present invention, the patient can be an adolescent between the ages of 12 and 18 years of age. In still other embodiments, the patient can be a child less than 12 years of age. In certain other embodiments, the patient can be a child less than 5 years of age. In still other embodiments, the patient can be a child between the ages of 2 and 12 years of age. In yet other embodiments, the patient can be a child between the ages of 12 months and 8 years of age. In other embodiments, the patient can be an infant less than 2 years of age.

In other embodiments of the present invention, the methods have a delivery time of less than about 5 minutes. In certain other embodiments of the present invention, the methods have a delivery time of less than about 4 minutes. In other embodiments, the methods have a delivery time of less than about 3 minutes. In still other embodiments, the methods have a delivery time of less than about 2 minutes. In yet other embodiments, the methods have a delivery time of less than about 1.5 minutes. In yet still another embodiment, the methods have a delivery time of less than about 1 minute. In one embodiment, the method has a delivery time of between about 30 seconds and about 1 minute. In another embodiment, the method has a delivery time of between about 1 minute and about 2 minutes. In yet another embodiment, the method has a delivery time of between about 2 minutes to about 3 minutes. In still another embodiment, the method has a delivery time of between about 1 minute and about 3 minutes. In still another embodiment, the method has a delivery time of between about 3 minutes to about 4 minutes.

In certain aspects of the present invention, the volume of the inhalation mixture comprising a β₂ agonist is less than 5.0 ml. In certain embodiments of the present invention, the volume of the inhalation mixture comprising a β₂ agonist is from about 0.1 ml to about 1.5 ml. In one embodiment, the volume of the inhalation mixture comprising a β₂ agonist is from about 0.1 ml to about 1.0 ml. In another embodiment, the volume of the inhalation mixture comprising a β₂ agonist is from about 0.3 ml to about 0.8 ml. In yet another embodiment, the volume of the inhalation mixture comprising a β₂ agonist is from about 0.4 ml to about 0.6 ml. In still yet another embodiment, the volume of the inhalation mixture comprising a β₂ agonist is about 0.5 ml.

In certain other embodiments of the present invention, the methods described herein can comprise the delivery of two or at least two, three or at least three, four or at least four, five or at least five, or six or at least six or more doses of an inhalation mixture comprising a β₂ agonist to a patient in need thereof.

In other embodiments of the present invention, the methods described herein are for the treatment of a patient diagnosed with, suspected of having, or experiencing the symptoms of a disorder selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

In certain embodiments of the present invention, the invention comprises a dosage formulation for administration by inhalation nebulization comprising less than about 0.21 mg of a β₂ agonist or a pharmaceutically acceptable salt thereof; whereby the formulation is suitable for delivery by an inhalation nebulizer and said delivery takes less than about 5 minutes. In certain embodiments, the dosage formulation can further comprise a preservative. In certain other embodiments, the dosage formulation can further comprise a solubility enhancer. In still other embodiments, the dosage formulation can comprise a preservative and/or a solubility enhancer and can further comprise a pharmaceutically acceptable excipient, and/or a chelating agent, a sequestering agent, or an antioxidant.

In other embodiments, the dosage formulations described herein are suitable for delivery by an inhalation nebulizer wherein the delivery time is less than about 4 minutes. In other embodiments, the dosage formulations described herein are suitable for delivery by an inhalation nebulizer wherein the delivery time is less than about 3 minutes. In still other embodiments, the dosage formulations described herein are suitable for delivery by an inhalation nebulizer wherein the delivery time is less than about 2 minutes. In yet other embodiments, the dosage formulations described herein are suitable for delivery by an inhalation nebulizer wherein the delivery time is less than about 1.5 minutes. In yet still another embodiment, the dosage formulations described herein are suitable for delivery by an inhalation nebulizer wherein the delivery time is less than about 1 minute. In one embodiment, the dosage formulations described herein are suitable for delivery by an inhalation nebulizer wherein the delivery time is between about 30 seconds and about 1 minute. In another embodiment, the dosage formulations described herein are suitable for delivery by an inhalation nebulizer wherein the delivery time is between about 1 minute and about 2 minutes. In yet another embodiment, the dosage formulations described herein are suitable for delivery by an inhalation nebulizer wherein the delivery time is between about 2 minutes to about 3 minutes. In still another embodiment, the method has a delivery time of between about 1 minute and about 3 minutes. In still yet another embodiment, the dosage formulations described herein are suitable for delivery by an inhalation nebulizer wherein the delivery time is between about 3 minutes to about 4 minutes.

In certain other embodiments of the present invention, the invention comprises a dosage formulation for administration by inhalation nebulization comprising: (a) a β₂ agonist or a pharmaceutically acceptable salt thereof; and (b) a preservative; whereby said formulation is suitable for delivery by an inhalation nebulizer and said delivery takes less than about 5 minutes. In certain embodiments, the dosage formulation can further comprise a solubility enhancer. In still other embodiments, the dosage formulation comprising a preservative and/or a solubility enhancer can further comprise a pharmaceutically acceptable excipient, and/or a chelating agent, a sequestering agent, or an antioxidant.

In certain embodiments of the present invention, the β₂ agonist is a short acting β₂ agonist selected from the group consisting of albuterol, albuterol free base, albuterol sulfate, albuterol hydrochloride, albuterol maleate, albuterol tartrate, albuterol citrate, albuterol phosphate, terbutaline sulfate, bitolterol mesylate, levalbuterol, metaproterenol sulfate, pirbuterol acetate, and combinations thereof.

In other embodiments, the short acting β₂ agonist is selected from the group consisting of albuterol, albuterol free base, albuterol sulfate, albuterol hydrochloride, albuterol maleate, albuterol tartrate, albuterol citrate, albuterol phosphate, terbutaline sulfate, levalbuterol, pirbuterol acetate, and combinations thereof.

In certain other embodiments, the short acting β₂ agonist is selected from the group consisting of albuterol, albuterol free base, albuterol sulfate, albuterol hydrochloride, albuterol citrate, albuterol phosphate, or levalbuterol.

In one embodiment, the short acting β₂ agonist is albuterol free base. In another embodiment, the short acting β₂ agonist is albuterol sulfate. In still another embodiment, the short acting β₂ agonist is albuterol hydrochloride. In still yet another embodiment, the short acting β₂ agonist is albuterol citrate. In yet still another embodiment, the short acting β₂ agonist is albuterol phosphate. In yet another embodiment, the short acting β₂ agonist is levalbuterol.

In certain other embodiments of the present invention, the β₂ agonist is a long acting β₂ agonist selected from the group consisting of formoterol, arformoterol, arformoterol tartrate, salmeterol, or combinations thereof.

In certain embodiments of the dosage formulation, the preservative is selected from the group consisting of edetate disodium (EDTA), benzalkonium chloride (BAC), and combinations thereof. In one embodiment, the preservative is EDTA. In another embodiment, the preservative is benzalkonium chloride.

In certain embodiments of the dosage formulation, the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE-γ-CD, hydroxypropyl-β-cyclodextrin, 2-HP-β-CD, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof.

In certain embodiments of the present invention, the dosage formulation described herein can further comprise a pharmaceutically acceptable excipient selected from the group consisting of a chelating agent, a sequestering agent, or an antioxidant.

In certain embodiments of the present invention, the dosage formulation describe herein further comprises a second pharmaceutically active agent. In one embodiment, the second pharmaceutically active agent is a corticosteroid. In another embodiment, the second pharmaceutically active agent is an antibiotic. In yet another embodiment, the second pharmaceutically active agent is an anti-cholinergic agent. In still another embodiment, the second pharmaceutically active agent is a dopamine (D₂) receptor agonist.

### INCORPORATION BY REFERENCE

Unless stated otherwise, all publications and patent applications mentioned in this specification are herein incorporated by reference to the same extent as if each individual publication or patent application was specifically and individually indicated to be incorporated by reference.

### BRIEF DESCRIPTION OF THE FIGURES

FIG. 1 is the anterior view of a lung scintigraph after administration of the following radio-labeled albuterol solutions: (a) 0.63mg/0.5 mL via Pari eFlow nebulizer; (b) 1.25 mg/0.5 mL via Pari eFlow nebulizer; and (c) Ventolin® (2.5 mg/3 mL) via Pari LC Plus nebulizer.

FIG. 2 is the anterior view of a lung scintigraph after administration of the following radio-labeled albuterol solutions: (a) 0.63 mg/0.5 mL via Pari eFlow nebulizer; (b) 1.25 mg/0.5 mL via Pari eFlow nebulizer; and (c) Ventolin ® (2.5 mg/3 mL) via Pari LC Plus nebulizer.

FIG. 3 is the anterior view of a lung scintigraph after administration of the following radio-labeled albuterol solutions: (a) 0.63 mg/0.5 mL via Pari eFlow nebulizer; (b) 1.25 mg/0.5 mL via Pari eFlow nebulizer; and (c) Ventolin ® (2.5 mg/3 mL) via Pari LC Plus nebulizer.

### DETAILED DESCRIPTION OF THE INVENTION

Reference will now be made in detail to embodiments of the methods and dosage formulations disclosed herein. Examples of the embodiments are illustrated in the following Examples section.

Unless defined otherwise, all technical and scientific terms used herein have the same meaning as is commonly understood by one of skill in the art to which the inventions described herein belong. All patents and publications referred to herein are incorporated by reference.

### Certain Definitions

As used herein, the terms “comprising,” “including,” “such as,” and “for example” are used in their open, non-limiting sense.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe.

“Albuterol” is an optically active compound which can exist as an (R)- or an (S)-enantiomer, or as a mixture of the two enantiomers. The term “albuterol” usually refers to a racemic mixture of both the (R)- and (S)-albuterol enantiomers. Herein, the term albuterol is defined as including a racemic mixture, a single enantiomer of albuterol, or any mixture of enantiomers of albuterol. Traditional racemic albuterol and racemic albuterol sulfate are commercially available as Proventil®, Ventolin® and Vormax®. The pure (R)-enantiomer, which has the generic name levalbuterol, is commercially available as Xopenex®. In addition, albuterol, as used herein, includes salbutamol, albuterol free base as well as pharmaceutically acceptable salts of albuterol, including, but not limited to, hydrochloride, sulfate, maleate, tartrate, citrate, phosphate and the like. Certain exemplary salts are described in U.S. Pat. No. 3,644,353, which is incorporated herein by reference in its entirety.

“Bioavailability”refers to the percentage of the weight of a β₂ agonist, such as albuterol, dosed that is delivered into the general circulation of the animal or human being studied. The total exposure (AUC(0-∞)) of a drug when administered intravenously is usually defined as 100% Bioavailable (F %).

“Bronchoconstrictive disorder,” as used herein, refers to any disorder or disease related to the reduction in the inner diameter of the bronchial pathway, e.g., a bronchus or bronchi, including, but not limited to, asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

“Bronchodilation,” as used herein, refers to the expansion of the bronchial air passages to treat or prevent a bronchoconstrictive disorder.

“β₂ agonists” or “β₂ adrenergic receptor agonists,” as used herein, refers to any agent which can activate the β₂ adrenergic receptor. Short acting or long acting β₂ agonists are known in the art and include, but are not limited to, albuterol (e.g., albuterol free base, albuterol sulfate, albuterol hydrochloride, albuterol maleate, albuterol tartrate, albuterol citrate, albuterol phosphate), terbutaline sulfate, bitolterol mesylate, levalbuterol, metaproterenol sulfate, pirbuterol acetate, formoterol, arformoterol, arformoterol tartrate, salmeterol, or combinations thereof In certain embodiments of the present invention, the dosage formulations comprising β₂ agonists are sterile, thus eliminating the need for preservatives. In other embodiments, the dosage formulations comprising β₂ agonists can comprise a preservative.

“Corticosteroids,” as used herein, refers to a group of drugs similar to the natural corticosteroid hormones produced by the cortex of the adrenal glands. Corticosteroids act to inhibit late phase allergic reactions via a variety of mechanisms, including decreasing the density of mast cells along mucosal surfaces, decreasing chemotaxis and activation of eosinophils, decreasing cytokine production by lymphocytes, monocytes, mast cells and eosinophils, inhibiting the metabolism of arachidonic acid and other mechanisms. Corticosteroids include, but are not limited to, for example, budesonide.

“Drug absorption” or “absorption” typically refers to the process of movement of drug from site of delivery of a drug across a barrier into a blood vessel or the site of action, e.g., a drug being absorbed in the pulmonary capillary beds of the alveoli.

“Equal,” as used herein, refers to two or more parameters or values having substantially the same value. As one of ordinary skill in the art would understand, the exact boundary of “equal” will depend on the particular parameter or value being analyzed. Illustratively, the use of the term “equal,” as used herein, encompasses values slightly outside the cited values, i.e., plus or minus 0.1% to 25%.

“Inhalation nebulizer,” as used herein, refers to a device that turns medications into a fine mist for delivery to the lungs.

“Inhalation mixture,” as used herein, refers to any dosage formulation for the inhaled delivery of an active agent. Examples of suitable inhalation mixtures include, but are not limited to, solutions, suspensions, dispersions, emulsions, colloidal liquids, micelle or mixed micelle liquids, and liposomal liquids. In some embodiments, the inhalation mixture is at least partially aqueous. In certain other embodiments, the inhalation mixture is substantially aqueous. In other embodiments, the inhalation mixture is a suspension formed by the introduction of a β₂ agonist in powder form into a solvent suitable for inhalation.

“Pharmacokinetics” refers to the factors which reflect the attainment and maintenance of the appropriate concentration of drug at a site of action.

“Preservatives,” as used herein, refers to any a chemical compound that is added to a dosage formulation to protect against decay or decomposition. As used herein, preservatives include chemical agents selected from the group of antimicrobials, antioxidants, complexing agents, and stabilizing agents. In certain embodiments, preservatives include, but are not limited to, edetate disodium (EDTA) or ethyleneglycol-bis(oxyethylenenitrilo)-tetraacetic acid (EGTA) and salts thereof, such as the disodium salt, citric acid, nitrilotriacetic acid, benzalkonium chloride (BAC) or benzoic acid, benzoates such as sodium benzoate, vitamins and vitamin esters, provitamins, ascorbic acid, vitamin E, and combinations thereof.

“Side effects,” as used herein, refers to potentially adverse effects of many β₂ agonist therapies. Such side effects include, but are not limited to, tremors, nervousness, shakiness, dizziness, increased appetite, and cardiac arrythmia. In children, side effects such as excitement, nervousness and hyperkinesia are also known. In certain embodiments, the methods of the present invention provide for the delivery of a therapeutically effect amount of a β₂ agonist to patient in need thereof at a concentration wherein the risks of side effects is reduced. In other embodiments, the methods of the present invention provide for the delivery of a therapeutically effect amount of a β₂ agonist to patient in need thereof at a concentration wherein the side effects are diminished or eliminated.

A “solubility enhancer,” as used herein, includes either chemical agents or methods of manufacturing which provide enhanced solubility of an active agent. In certain embodiments, “solubility enhancer” can refer to a chemical agent that, when present in the formulation, increases the solubility of a second chemical compound, such as an active ingredient, in a solvent, but which chemical compound is not itself a solvent for the second chemical compound. In other embodiments, “solubility enhancer” can refer to a formulation method which provides enhanced solubility without a chemical agent acting as the means to increase solubility.

A “therapeutically effective amount” or “effective amount” is that amount of a pharmaceutical agent to achieve a pharmacological effect. The term “therapeutically effective amount” includes, for example, a prophylactically effective amount. An “effective amount” of a β₂ agonist, such as albuterol, is an amount effective to achieve a desired pharmacologic effect or therapeutic improvement without undue adverse side effects. The effective amount of a β₂ agonist, such as albuterol, will be selected by those skilled in the art depending on the particular patient and the disease level. It is understood that “an effect amount” or “a therapeutically effective amount” can vary from subject to subject, due to variation in metabolism of a β₂ agonist, such as albuterol, age, weight, general condition of the subject, the condition being treated, the severity of the condition being treated, and the judgment of the prescribing physician.

“Treat” or “treatment” as used in the context of a bronchoconstrictive disorder refers to any treatment of a disorder or disease related to the constriction of the bronchi, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

I. Methods for the Delivery of a β₂ Agonist to Induce Bronchodilation

In certain aspects of the present invention, the methods described herein provide for delivery of a β₂ agonist to treat a disease by inducing bronchodilation in a patient in need thereof comprising the steps of (a) providing at least one dose of an inhalation mixture comprising a β₂ agonist to said patient; and (b) delivering said inhalation mixture with an inhalation nebulizer for less than about 5 minutes, wherein a therapeutically effective respirable dose of the β₂ agonist is provided to the patient at a concentration lower than the traditional β₂ agonist therapies. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet other embodiments, the β₂ agonist is administered at a concentration of less than about 1.0 mg/dose. In yet still other embodiments, the β₂ agonist is administered at a concentration of less than about 1.25 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In certain other aspects of the present invention, the methods described herein provide for delivery of a β₂ agonist to treat a disease by inducing bronchodilation in a patient in need thereof comprising the steps of (a) providing at least one dose of an inhalation mixture comprising a β₂ agonist to said patient; and (b) delivering said inhalation mixture with an inhalation nebulizer for less than about 5 minutes, wherein equivalent bronchodilation is provided to the patient at a concentration lower than traditional β₂ agonist therapies. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet still other embodiments, the β₂ agonist is administered at a concentration of less than about 1.25 mg/dose. In yet other embodiments, the β₂ agonist is administered at a concentration of less than about 1.0 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In still other aspects of the present invention, the methods described herein provide for delivery of a β₂ agonist to treat a disease by inducing bronchodilation in a patient in need thereof comprising the steps of (a) providing at least one dose of an inhalation mixture comprising a β₂ agonist to said patient; and (b) delivering said inhalation mixture with an inhalation nebulizer for less than about 5 minutes, wherein bronchodilation is achieved in a patient at a concentration lower than traditional β₂ agonist therapies. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet still other embodiments, the β₂ agonist is administered at a concentration of less than about 1.25 mg/dose. In yet other embodiments, the β₂ agonist is administered at a concentration of less than about 1.0 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

Further aspects of the present invention comprise methods for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein the methods reduce the risk of side effects associated with β₂ agonist treatment as compared to traditional β₂ agonist therapies. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet still other embodiments, the β₂ agonist is administered at a concentration of less than about 1.25 mg/dose. In yet other embodiments, the β₂ agonist is administered at a concentration of less than about 1.0 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In still other aspects of the present invention, the methods described herein provide for delivery of a β₂ agonist to treat a disease by inducing bronchodilation in a patient in need thereof comprising the steps of (a) providing at least one dose of an inhalation mixture comprising a β₂ agonist to said patient; and (b) delivering said inhalation mixture with an inhalation nebulizer for less than about 5 minutes, wherein bronchodilation is achieved in a patient at a concentration lower than the traditional β₂ agonist therapies. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet still other embodiments, the β₂ agonist is administered at a concentration of less than about 1.25 mg/dose. In yet other embodiments, the β₂ agonist is administered at a concentration of less than about 1.0 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In still another embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein at least about 20% of the β₂ agonist is deposited in the lung. In one embodiment, at least about 25% of the β₂ agonist is deposited in the lung. In yet another embodiment, at least about 30% of the β₂ agonist is deposited in the lung. In another embodiment, at least about 35% of the β₂ agonist is deposited in the lung. In yet another embodiment, at least about 40% of the β₂ agonist is deposited in the lung. In still another embodiment, at least about 50% of the β₂ agonist is deposited in the lung. In another embodiment, at least about 60% of the β₂ agonist is deposite in the lung. In yet another embodiment, at least about 80% of the β₂ agonist is deposited in the lung. In still yet another embodiment, between about 30% and about 60% of the β₂ agonist is deposited in the lung. In another embodiment, between about 30% and about 50% of the β₂ agonist is deposited in the lung. In yet another embodiment, between about 30% and about 40% of the β₂ agonist is deposited in the lung. In still another embodiment, between about 40% and about 50% of the β₂ agonist is deposited in the lung. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet still other embodiments, the β₂ agonist is administered at a concentration of less than about 1.25 mg/dose. In yet other embodiments, the β₂ agonist is administered at a concentration of less than about 1.0 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In still another embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein said method provides equivalent bronchodilation as compared to traditional β₂ agonist treatments at a β₂ agonist dose lower than traditional β₂ agonist treatments. In certain embodiments of the present invention, the β₂ agonist is administered at a concentration of less than about 2.5 mg/dose. In other embodiments, the β₂ agonist is administered at a concentration of less than about 2.0 mg/dose. In still other embodiments, the β₂ agonist is administered at a concentration of less than about 1.5 mg/dose. In yet still other embodiments, the β₂ agonist is administered at a concentration of less than about 1.25 mg/dose. In yet other embodiments, the β₂ agonist is administered at a concentration of less than about 1.0 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In still another embodiment of the present invention, the invention comprises a method for treating a disease by inducing bronchodilation in a patient in need thereof comprising: (a) providing at least one dose of an inhalation mixture comprising a β₂ agonist; and (b) delivering the inhalation mixture with an inhalation nebulizer for less than about 5 minutes, wherein said method provides greater bronchodilation as compared to traditional β₂ agonist treatments, wherein the β₂ agonist dose in the present invention is the same as the β₂ agonist dose in the traditional β₂ agonist treatments.

In one embodiment of the present invention, the invention comprises a method for treating a disease by inducing bronchodilation in a patient in need thereof comprising: (a) providing at least one dose of an inhalation mixture comprising a β₂ agonist to said patient; and (b) delivering said inhalation mixture with an inhalation nebulizer, wherein the β₂ agonist is provided at a concentration of less than about 0.21 mg/dose and whereby the delivering with said inhalation nebulizer is for less than about 5 minutes.

In another embodiment of the present invention, the invention comprises a method for treating a disease by inducing bronchodilation in a patient in need thereof comprising: (a) providing at least one dose of an inhalation mixture comprising a β₂ agonist at a concentration of less than about 0.21 mg/dose to said patient; and (b) delivering the inhalation mixture with an inhalation nebulizer for less than about 5 minutes, wherein at least about 20% of the β₂ agonist is deposited in the lung. In one embodiment, at least about 30% of the β₂ agonist is deposited in the lung. In another embodiment, at least about 40% of the β₂ agonist is deposited in the lung. In still another embodiment, at least about 50%, of the β₂ agonist is deposited in the lung. In yet still another embodiment, at least about 60% of the β₂ agonist is deposited in the lung. In another embodiment, at least about 70% of the β₂ agonist is deposited in the lung. In yet another embodiment, at least about 80% of the β₂ agonist is deposited in the lung.

In yet another embodiment of the present invention, the invention comprises a method for treating a disease by inducing bronchodilation in a patient in need thereof comprising: (a) providing at least one dose of an inhalation mixture comprising a β₂ agonist to said patient; and (b) delivering the inhalation mixture with an inhalation nebulizer for less than about 5 minutes, wherein less than about 30% of the β₂ agonist is delivered outside of the lung. In one embodiment, less than about 25% of the β₂ agonist is delivered outside of the lung. In another embodiment, less than about 20% of the β₂ agonist is delivered outside of the lung. In yet another embodiment, less than about 15% of the β₂ agonist is delivered outside of the lung. In still another embodiment, less than about 10% of the β₂ agonist is delivered outside of the lung. In yet still another embodiment, less than about 5% of the β₂ agonist is delivered outside the lung.

In certain embodiments, the β₂ agonist is administered at a concentration of less than about 0.21 mg/dose. In certain other embodiments of the present invention, the β₂ agonist is administered at a concentration of less than about 0.18 mg/dose. In other embodiments, the β₂ agonist is administered at a concentration of less than about 0.16 mg/dose. In still other embodiments, the β₂ agonist is administered at a concentration of less than about 0.14 mg/dose. In yet other embodiments, the β₂ agonist is administered at a concentration of less than about 0.12 mg/dose. In still yet other embodiments, the β₂ agonist is administered at a concentration of less than about 0.10 mg/dose. In one embodiment, the β₂ agonist is administered at a concentration of less than about 0.08 mg/dose. In another embodiment, the β₂ agonist is administered at a concentration of less than about 0.06 mg/dose. In yet another embodiment, the β₂ agonist is administered at a concentration of less than about 0.04 mg/dose. In other embodiments, the β₂ agonist is administered at a concentration of between about 0.04 mg/dose to about 0.1 mg/dose. In certain other embodiments, the β₂ agonist is administered at a concentration of between about 0.1 mg/dose to about 0.5 mg/dose. In still other embodiments, the β₂ agonist is administered at a concentration of between about 0.6 mg/dose to about 1.0 mg/dose. In yet other embodiments, the β₂ agonist is administered at a concentration of between about 1.0 mg/dose to about 1.5 mg/dose. In still yet other embodiments, the β₂ agonist is administered at a concentration of between about 1.5 mg/dose to about 2.0 mg/dose. In other embodiments, the β₂ agonist is administered at a concentration of less than about 2.0 mg/dose. In still other embodiments, the β₂ agonist is administered at a concentration of less than about 1.5 mg/dose. In yet other embodiments, the β₂ agonist is administered at a concentration of less than about 1.0 mg/dose. In yet still other embodiments, the β₂ agonist is administered at a concentration of less than about 1.25 mg/dose. In still yet other embodiments, the β₂ agonist is administered at a concentration of between about 0.04 mg/dose and about 1.5 mg/dose. In certain other embodiments, the β₂ agonist is administered at a concentration of between about 0.06 mg/dose and about 1.25 mg/dose. In other embodiments, the β₂ agonist is administered at a concentration of about 0.15 mg/dose. In certain other embodiments, the β₂ agonist is administered at a concentration of about 0.6 mg/dose. In still other embodiments, the β₂ agonist is administered at a concentration of about 1.25 mg/dose.

In other embodiments of the present invention, the invention comprises methods for treating a disease by inducing bronchodilation in a patient in need thereof comprising: (a) providing at least one dose of an inhalation mixture comprising a β₂ agonist to said patient; and (b) delivering said inhalation mixture with an inhalation nebulizer, wherein the β₂ agonist is provided at a concentration of between about 0.04 mg/dose to about 0.1 mg/dose and whereby said delivering with said inhalation nebulizer is for less than about 5 minutes. In certain embodiments, the β₂ agonist is provided at a concentration of between about 0.1 mg/dose to about 0.5 mg/dose and whereby said delivering with said inhalation nebulizer is for less than about 5 minutes. In other embodiments, the β₂ agonist is provided at a concentration of between about 0.6 mg/dose to about 1.0 mg/dose and whereby said delivering with said inhalation nebulizer is for less than about 5 minutes. In still other embodiments, the β₂ agonist is provided at a concentration of between about 1.0 mg/dose to about 1.5 mg/dose and whereby said delivering with said inhalation nebulizer is for less than about 5 minutes. In yet still other embodiments, the β₂ agonist is provided at a concentration of between about 1.5 mg/dose to about 2.0 mg/dose and whereby said delivering with said inhalation nebulizer is for less than about 5 minutes. In still yet other embodiments, the β₂ agonist is provided at a concentration of between about 0.04 mg/dose and about 1.5 mg/dose and whereby said delivering with said inhalation nebulizer is for less than about 5 minutes. In certain other embodiments, the β₂ agonist is provided at a concentration of between about 0.06 mg/dose and about 1.25 mg/dose and whereby said delivering with said inhalation nebulizer is for less than about 5 minutes. In other embodiments, the β₂ agonist is provide at a concentration of about 0.15 mg/dose and whereby said delivering with said inhalation nebulizer is for less than about 5 minutes. In certain other embodiments, the β₂ agonist is provided at a concentration of about 0.6 mg/dose and whereby said delivering with said inhalation nebulizer is for less than about 5 minutes. In still other embodiments, the β₂ agonist is provided at a concentration of about 1.25 mg/dose and whereby said delivering with said inhalation nebulizer is for less than about 5 minutes.

In other embodiments of the present invention, the methods have a delivery time of less than about 4 minutes. In other embodiments, the methods have a delivery time of less than about 3 minutes. In still other embodiments, the methods have a delivery time of less than about 2 minutes. In yet other embodiments, the methods have a delivery time of less than about 1.5 minutes. In yet still another embodiment, the methods have a delivery time of less than about 1 minute. In one embodiment, the method has a delivery time of between about 30 seconds and about 1 minute. In another embodiment, the meth6d has a delivery time of between about 1 minute and about 2 minutes. In yet another embodiment, the method has a delivery time of between about 2 minutes to about 3 minutes. In still another embodiment, the method has a delivery time of between about 1 minute and about 3 minutes. In still yet another embodiment, the method has a delivery time of between about 3 minutes to about 4 minutes.

In certain embodiments of the present invention, the patient can be an adult over 18 years of age. In other embodiments of the present invention, the patient can be an adolescent between the ages of 12 and 18 years of age. In still other embodiments, the patient can be a child less than 12 years of age. In certain other embodiments, the patient can be a child less than 5 years of age. In still other embodiments, the patient can be a child between the ages of 2 and 12 years of age. In yet other embodiments, the patient can be a child between the ages of 12 months and 8 years of age. In other embodiments, the patient can be an infant less than 2 years of age.

In certain aspects of the present invention, the volume of the inhalation mixture comprising a β₂ agonist is less than 5.0 ml. In certain embodiments of the present invention, the volume of the inhalation mixture comprising a β₂ agonist is from about 0.1 ml to about 1.5 ml. In one embodiment, the volume of the inhalation mixture comprising a β₂ agonist is from about 0.1 ml to about 1.0 ml. In another embodiment, the volume of the inhalation mixture comprising a β₂ agonist is from about 0.3 ml to about 0.8 m-d. In yet another embodiment, the volume of the inhalation mixture comprising a β₂ agonist is from about 0.4 ml to about 0.6 ml. In still yet another embodiment, the volume of the inhalation mixture comprising a β₂ agonist is about 0.5 ml.

In other embodiments of the present invention, the invention can comprise methods for treating a disease by inducing bronchodilation in a patient in need thereof comprising the consecutive delivery of more than one dose of an inhalation mixture comprising a β₂ agonist with an inhalation nebulizer, wherein each delivery by an inhalation nebulizer is for less than about 5 minutes. In one embodiment, the method comprises the consecutive delivery of at least two doses of an inhalation mixture comprising a β₂ agonist to a patient in need thereof. In another embodiment, the method comprises the consecutive delivery of at least three doses of an inhalation mixture comprising a β₂ agonist to a patient in need thereof. In still another embodiment, the method comprises the consecutive delivery of at least four doses of an inhalation mixture comprising a β₂ agonist to a patient in need thereof. In yet still another embodiment, the method comprises the consecutive delivery of at least five doses of an inhalation mixture comprising a β₂ agonist to a patient in need thereof. In another embodiment, the method comprises the consecutive delivery of at least six doses of an inhalation mixture comprising a β₂ agonist to a patient in need thereof. In some embodiments, the method comprises the consecutive delivery of six or more doses of an inhalation mixture comprising a β₂ agonist to a patient in need thereof.

In certain embodiments of the present invention, the β₂ agonist is a short acting β₂ agonist selected from t group consisting of albuterol (e.g., albuterol free base, albuterol sulfate, albuterol hydrochloride, albuterol maleate, albuterol tartrate, albuterol citrate, albuterol phosphate), terbutaline sulfate, bitolterol mesylate, levalbuterol, metaproterenol sulfate, pirbuterol acetate, and combinations thereof. In other embodiments, the short acting β₂ agonist is selected from the group consisting of albuterol, albuterol free base, albuterol sulfate, albuterol hydrochloride, albuterol maleate, albuterol tartrate, albuterol citrate, albuterol phosphate, terbutaline sulfate, levalbuterol, pirbuterol acetate, and combinations thereof. In certain other embodiments, the short acting β₂ agonist is selected from the group consisting of albuterol, albuterol free base, albuterol sulfate, albuterol hydrochloride, albuterol citrate, albuterol phosphate or levalbuterol.

In one embodiment, the short acting β₂ agonist is albuterol free base. In another embodiment, the short acting β₂ agonist is albuterol sulfate. In still another embodiment, the short acting β₂ agonist is albuterol hydrochloride. In still yet another embodiment, the short acting β₂ agonist is albuterol citrate. In yet still another embodiment, the short acting β₂ agonist is albuterol phosphate. In yet another embodiment, the short acting β₂ agonist is levalbuterol.

In certain other embodiments of the present invention, the β₂ agonist is a long acting β₂ agonist selected from the group consisting of formoterol, arformoterol, arformoterol tartrate, salmeterol, or combinations thereof.

Combination Therapies

In other embodiments of the present invention, the methods can further comprise a combination therapy wherein the delivery of an inhalation mixture comprising a β₂ agonist further comprises the delivery of a second pharmaceutically active agent by an inhalation nebulizer. In some embodiments, the combination therapy comprises an inhalation mixture comprising a β₂ agonist and a second pharmaceutically active agent wherein the β₂ agonist and a second pharmaceutically active agent are delivered simultaneously. In other embodiments, the combination therapy can comprise an inhalation mixture comprising a β₂ agonist and an inhalation mixture comprising a second pharmaceutically active agent wherein the two inhalation mixtures are simultaneously delivered. In other embodiments, the combination therapy comprises an inhalation mixture comprising a β₂ agonist and the inhalation mixture comprising a second pharmaceutically active agent wherein the two inhalation mixtures are consecutively delivered.

In certain embodiments, the combination therapies described herein include the delivery of an inhalation mixture comprising a β₂ agonist and a second pharmaceutically active agent selected from (a) a corticosteroid; (b) an antibiotic; (c) an anti-cholinergic agent; or (d) a dopamine (D₂) receptor agonist.

Corticosteriods for use in the combination therapies herein include, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In one embodiment, the second pharmaceutically active agent is budesonide.

Antibiotics for use in the combination therapies described herein include, but are not limited to, penicillins, cephalosporins, macrolides, sulfonamides, aminoglycosides, and β-lactam antibiotics.

Anticholinergic agents for use herein include, but are not limited to, ipratropium bromide, oxitropium bromide, atropine methyl nitrate, atropine sulfate, ipratropium, belladonna extract, scopolamine, scopolamine methobromide, homatropine methobromide, hyoscyamine, isopriopramide, orphenadrine, tiotropium bromide and glycopyrronium bromide.

Dopamine (D₂) receptor agonists for use in the combination therapy described herein include, but are not limited to, Apomorphine ((r)-5,6,6a,7-tetrahydro-6-methyl-4H-dibenzo[de,g]quinoline-10,11-diol); Bromocriptine ((5′α)-2-bromo-12′-hydroxy-2′-(1-methylethyl)-5′-(2-methylpropyl)ergotaman-3′,6′,18-trione); Cabergoline ((8β)-N-(3(dimethylamino)propyl)-N-((ethylamino)carbonyl)6-(2-propeny 1)ergoline-8-carboxamide); Lisuride (N′-((8 α)-9,10-didehydro-6-methylergolin-8-yl)-N,N-diethylurea); Pergolide ((8β)-8-((methylthio)methyl)-6-propylergoline); Levodopa (3-hydroxy-L-tryrosine); Pramipexole ((s)-4,5,6,7-tetrahydro-N₆-propyl-2,6-benzothiazolediamine); Quinpirole hydrochloride (trans-(−)4aR-4,4a,5,6,7,8,8a,9-octahydro-5-propyl-1H-pyrazolo[3,4-g] quinoline hydrochloride); Ropimrole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine). Other dopamine D₂ receptor agonists for use herein are disclosed in International Patent Application Publication No. WO 99/36095.

Other active ingredients for use in the inhalable compositions described herein include, but are not limited to, IL-5 inhibitors such as those disclosed in U.S. Pat. No. 5,668,110, No. 5,683,983, No. 5,677,280, No. 6,071,910 and No. 5,654,276, each of which is incorporated by reference herein; anti-sense modulators of IL-5 such as those disclosed in U.S. Pat. No. 6,136,603, the relevant disclosure of which is hereby incorporated by reference; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623, which is incorporated by reference herein; tachykinin receptor antagonists such as those disclosed in U.S. Pat. No. 5,691,336, No. 5,877,191, No. 5,929,094, No. 5,750,549 and No. 5,780,467, each of which is incorporated by reference herein; leukotriene receptor antagonists such as montelukast sodium (Singular, R-(E)]-1-[[[1-[3-[2-(7-chloro-2-quinolinyl)-ethenyl]-phenyl]-3-[2-(I-hydroxy-1-methylethyl)-phenyl]-propyl]-thio]-methyl] cyclopro-paneacetic acid, monosodium salt), 5-lypoxygenase inhibitors such as zileuton (Zyflo®, Abbott Laboratories, Abbott Park, Ill.), and anti-IgE antibodies such as Xolair (recombinant humanized anti-IgE monoclonal antibody (CGP 51901; IGE 025A; rhuMAb-E25), Genentech, Inc., South San Francisco, Calif.), and topical anesthetics such as lidocaine, N-arylamide, aminoalkylbenzoate, prilocaine, etidocaine (U.S. Pat. No. 5,510,339, No. 5,631,267, and No. 5,837,713, the relevant disclosures of which are hereby incorporated by reference).

For illustrative purposes only, and not to be construed as a limitation thereon, certain embodiments of the present invention can include: (a) providing at least one dose of a dosage formulation of an inhalation mixture comprising less than 0.21 mg of albuterol; (b) adding a second pharmaceutically active agent, e.g., Pulmicort® Respules (an aqueous suspension comprising budesonide) to the inhalation mixture; and (c) delivering the inhalation mixture comprising (a) and (b) to a patient in need thereof with an inhalation nebulizer for less than about 5 minutes. In one embodiment, (a) is initially added to the nebulizer and then (b) is added to the nebulizer and the inhalation mixture comprising (a) and (b) is delivered simultaneously. In another embodiment, (b) is initially added to the nebulizer and then (a) is added to the nebulizer and the inhalation mixture comprising (a) and (b) is delivered simultaneously. In yet another embodiment, (a) and (b) are simultaneously added to the nebulizer, and the inhalation mixture comprising (a) and (b) is delivered simultaneously. In another embodiment, (a) and (b) are delivered consecutively with either (a) or (b) delivered first. In alternate embodiments, the albuterol is administered at a concentration of less than about 0.18 mg/dose. In other embodiments, the albuterol is administered at a concentration of less than about 0.16 mg/dose. In still other embodiments, the albuterol is administered at a concentration of less than about 0.14 mg/dose. In yet other embodiments, the albuterol is administered at a concentration of less than about 0.12 mg/dose. In still yet other embodiments, the albuterol is administered at a concentration of less than about 0.10 mg/dose. In one embodiment, the albuterol is administered at a concentration of less than about 0.08 mg/dose. In another embodiment, the albuterol is administered at a concentration of less than about 0.06 mg/dose. In yet another embodiment, the albuterol is administered at a concentration of less than about 0.04 mg/dose.

In still other embodiments, the present invention can include: (a) providing at least one dose dosage formulation of an inhalation mixture comprising less than 0.21 mg of albuterol and a solubility enhancer, e.g., SBE-β-CD (Captisol® (Cydex, Inc. Lenexa, Kans.); (b) adding a second pharmaceutically active agent, e.g., Pulmicort® Respules (an aqueous suspension comprising budesonide) to the inhalation mixture; and (c) delivering the inhalation mixture comprising (a) and (b) to a patient in need thereof with an inhalation nebulizer for less than about 5 minutes. In one embodiment, (a) is initially added to the nebulizer and then (b) is added to the nebulizer, and the inhalation mixture comprising (a) and (b) is delivered simultaneously. In another embodiment, (b) is initially added to the nebulizer and then (a) is added to the nebulizer, and the inhalation mixture comprising (a) and (b) is delivered simultaneously. In yet another embodiment, (a) and (b) are simultaneously added to the nebulizer, and the inhalation mixture comprising (a) and (b) is delivered simultaneously. In another embodiment, (a) and (b) are delivered consecutively, with either (a) or (b) delivered first. In alternate embodiments, the albuterol is administered at a concentration of less than about 0.18 mg/dose. In other embodiments, the albuterol is administered at a concentration of less than about 0.16 mg/dose. In still other embodiments, the albuterol is administered at a concentration of less than about 0.14 mg/dose. In yet other embodiments, the albuterol is administered at a concentration of less than about 0.12 mg/dose. In still yet other embodiments, the albuterol is administered at a concentration of less than about 0.10 mg/dose. In one embodiment, the albuterol is administered at a concentration of less than about 0.08 mg/dose. In another embodiment, the albuterol is administered at a concentration of less than about 0.06 mg/dose. In yet another embodiment, the albuterol is administered at a concentration of less than about 0.04 mg/dose.

In other embodiments of the present invention, the methods described herein are for the treatment of a patient diagnosed with, or suspected of having, a disorder or disease selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

II. β₂ Agonist Formulations for Use in the Present Methods

In other aspects of the present invention, the dosage formulations for administration by the methods described herein comprise: (a) less than about 0.21 mg of a β₂ agonist or a pharmaceutically acceptable salt thereof; whereby said formulation is suitable for delivery by an inhalation nebulizer and said delivery takes less than about 5 minutes. In certain embodiments, the dosage formulations can further comprise a preservative. In certain other embodiments, the dosage formulations can further comprise a solubility enhancer, and/or a chelating agent, a sequestering agent, or an antioxidant.

In certain embodiments of the dosage formulation described herein, the β₂ agonist is less than about 0.18 mg/dose. In other embodiments, the β₂ agonist is less than about 0.16 mg/dose. In still other embodiments, the β₂ agonist is less than about 0.14 mg/dose. In yet other embodiments, the β₂ agonist is less than about 0.12 mg/dose. In still yet other embodiments, the β₂ agonist is less than about 0.10 mg/dose. In one embodiment, the β₂ agonist is of less than about 0.08 mg/dose. In another embodiment, the β₂ agonist is less than about 0.06 mg/dose. In yet another embodiment, the β₂ agonist is less than about 0.04 mg/dose.

In certain embodiments, the dosage formulations described herein are suitable for delivery by an inhalation nebulizer wherein the delivery time is less than about 4 minutes. In other embodiments, the dosage formulations described herein are suitable for delivery by an inhalation nebulizer wherein the delivery time is less than about 3 minutes. In still other embodiments, the dosage formulations described herein are suitable for delivery by an inhalation nebulizer wherein the delivery time is less than about 2 minutes. In yet other embodiments, the dosage formulations described herein are suitable for delivery by an inhalation nebulizer wherein the delivery time is less than about 1.5 minutes. In yet still another embodiment, the dosage formulations described herein are suitable for delivery by an inhalation nebulizer wherein the delivery time is less than about 1 minute. In one embodiment, the dosage formulations described herein are suitable for delivery by an inhalation nebulizer wherein the delivery time is between about 30 seconds and about 1 minute. In another embodiment, the dosage formulations described herein are suitable for delivery by an inhalation nebulizer wherein the delivery time is between about 1 minute and about 2 minutes. In yet another embodiment, the dosage formulations described herein are suitable for delivery by an inhalation nebulizer wherein the delivery time is between about 2 minutes to about 3 minutes. In still another embodiment, the method has a delivery time of between about 1 minute and about 3 minutes. In still yet another embodiment, the dosage formulations described herein are suitable for delivery by an inhalation nebulizer wherein the delivery time is between about 3 minutes to about 4 minutes.

In still other aspects of the present invention, the dosage formulations for administration by the methods described herein comprise: (a) a β₂ agonist or a pharmaceutically acceptable salt thereof; and (b) a preservative; whereby said formulation is suitable for delivery by an inhalation nebulizer and said delivery takes less than about 5 minutes. In certain embodiments, the dosage formulation can further comprise a solubility enhancer, and/or a chelating agent, a sequestering agent, or an antioxidant.

In certain aspects, the dosage formulations described herein are suitable for delivery by an inhalation nebulizer wherein the delivery time is less than about 4 minutes. In other embodiments, the dosage formulations described herein are suitable for delivery by an inhalation nebulizer wherein the delivery time is less than about 3 minutes. In still other embodiments, the dosage formulations described herein are suitable for delivery by an inhalation nebulizer wherein the delivery time is less than about 2 minutes. In yet other embodiments, the dosage formulations described herein are suitable for delivery by an inhalation nebulizer wherein the delivery time is less than about 1.5 minutes. In yet still another embodiment, the dosage formulations described herein are suitable for delivery by an inhalation nebulizer wherein the delivery time is less than about 1 minute. In one embodiment, the dosage formulations described herein are suitable for delivery by an inhalation nebulizer wherein the delivery time is between about 30 seconds and about 1 minute. In another embodiment, the dosage formulations described herein are suitable for delivery by an inhalation nebulizer wherein the delivery time is between about 1 minute and about 2 minutes. In yet another embodiment, the dosage formulations described herein are suitable for delivery by an inhalation nebulizer wherein the delivery time is between about 2 minutes to about 3 minutes. In still another embodiment, the method has a delivery time of between about 1 minute and about 3 minutes. In still yet another embodiment, the dosage formulations described herein are suitable for delivery by an inhalation nebulizer wherein the delivery time is between about 3 minutes to about 4 minutes.

In other aspects of the present invention, the dosage formulations comprising a β₂ agonist include described herein include, but are not limited to, solutions, dispersions, emulsions, colloidal liquids, micelle or mixed micelle solutions, and liposomal liquids. In one embodiment, the dosage formulation is a solution comprising less than about 0.21 mg of a β₂ agonist, such as albuterol. In another embodiment, the dosage formulation is a mixed micelle solution comprising less than about 0.21 mg of a β₂ agonist, such as albuterol. In yet another embodiment, the dosage formulation is a liposomal solution comprising less than about 0.21 mg of a β₂ agonist, such as albuterol.

In certain other embodiments, the dosage formulation is a solution comprising a β₂ agonist, such as albuterol, and a preservative. In another embodiment, the dosage formulation is a mixed micelle solution comprising a β₂ agonist, such as albuterol, and a preservative. In yet another embodiment, the dosage formulation is a liposomal solution comprising a β₂ agonist, such as albuterol, and a preservative. In certain embodiments, the dosage formulations described herein can further comprise a solubility enhancer. In certain other embodiments, the dosage formulations described herein can further comprise a pharmaceutically acceptable excipient, and/or a chelating agent, a sequestering agent, or an antioxidant.

β₂ agonists useful in dosage formulations of the present invention include, but are not limited to, short acting β₂ agonists selected from the group consisting of albuterol (e.g., albuterol free base, albuterol sulfate, albuterol hydrochloride, albuterol maleate, albuterol tartrate, albuterol citrate, albuterol phosphate), terbutaline sulfate, bitolterol mesylate, levalbuterol, metaproterenol sulfate, pirbuterol acetate, and combinations thereof. In other embodiments, the short acting β₂ agonist is selected from the group consisting of albuterol, albuterol free base, albuterol sulfate, albuterol hydrochloride, albuterol maleate, albuterol tartrate, albuterol citrate, albuterol phosphate, terbutaline sulfate, levalbuterol, pirbuterol acetate, and combinations thereof.

In certain other embodiments, the short acting β₂ agonist is selected from the group consisting of albuterol, albuterol free base, albuterol sulfate, albuterol hydrochloride, albuterol citrate, albuterol phosphate, or levalbuterol. In one embodiment, the short acting β₂ agonist is albuterol free base. In another embodiment, the short acting β₂ agonist is albuterol sulfate. In still another embodiment, the short acting β₂ agonist is albuterol hydrochloride. In yet still another embodiment, short acting β₂ agonist is albuterol citrate. In still yet another embodiment, short acting β₂ agonist is albuterol phosphate. In yet another embodiment, the short acting β₂ agonist is levalbuterol.

In other embodiments of the present invention, the β₂ agonist useful in dosage formulations of the present invention include long acting β₂ agonists including, but not limited to, formoterol, arformoterol, arformoterol tartrate, salmeterol, or combinations thereof.

In certain embodiments of the present invention, the dosage formulation comprising a β₂ agonist further comprises a preservative. Preservatives include any a chemical compound that is added to a dosage formulation to protect against decay or decomposition and include antimicrobial agents, antioxidants, complexing agents, and stabilizing agents. In some embodiments, the preservative can have a concentration (w/v) ranging from about 0.001% to about 5%. Suitable preservatives for use in the dosage formulations described herein include, but are not limited to, edetate disodium (EDTA) or ethyleneglycolbis(oxyethylenenitrilo)-tetraacetic acid (EGTA) and salts thereof, such as the disodium salt, citric acid, nitrilotriacetic acid, benzalkonium chloride (BAC) or benzoic acid, benzoates such as sodium benzoate, vitamins and vitamin esters, provitamins, ascorbic acid, vitamin E, and combinations thereof. In one embodiment, the formulation comprising a β₂ agonist further comprises edetate disodium (EDTA). In another embodiment, the formulation comprising a β₂ agonist further comprises benzalkonium chloride (BAC).

In some embodiments of the dosage formulation described herein, dosage formulation comprising a β₂ agonist further comprises a solubility enhancer. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In one embodiment, the solubility enhancer has a concentration (w/v) ranging from about 5% to about 10% wherein the solubility enhancer is a cyclodextrin or cyclodextrin derivative.

Solubility enhancers suitable for use in the dosage formulation of the present invention include, but are not limited to, propylene glycol, non-ionic surfactants, phospholipids, cyclodextrins and derivatives thereof, and surface modifiers and/or stabilizers. Solubility enhancers are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Examples of non-ionic surfactants which appear to have a particularly good physiological compatibility for use in the dosage formulation of the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the tradename Tweens 20-40-60, etc.), Polysorbate 80, Polyethylene glycol 400; sodium lauryl sulfate; sorbitan laurate, sorbitan palmitate, sorbitan stearate (available under the tradename Span 20-40-60 etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the dosage formulation of the present invention are formulated with the β₂ agonist to form liposome preparations, micelles or mixed micelles. Methods for the preparations and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vemuri et al. (Preparation and characterization of liposomes as therapeutic delivery systems: a review. Pharm Acta Helv. 1995, 70(2):95-1 11) and U.S. Pat. Nos. 5,019,394, 5,192,228, 5,882,679, 6,656,497 each of which is specifically incorporated by reference herein.

16] In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. Both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solublized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 nm to about 100 nm. Particularly preferred are micelles with average diameters of about 10 to about 50 nm. Methods of producing micelles and mixed micelles are known in the art and described in, for example, U.S. Pat. Nos. 5,747,066 and 6,906,042, each of which is specifically incorporated by reference herein.

Phospholipids are defined as amphiphile lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, tryglycerides and other substances.

Additional phospholipids which are suitable for delivery by inhalation on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phopholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the β₂ agonist to form colloidal structures. Colloidal solutions are defined as mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, β-cyclodextrins, γ-cyclodextrins, SAE-CD derivatives (e.g., SBE-α-CD, SBE-β-CD (Captisol®, Cydex, Inc. Lenexa, Kans.), and SBE-γ-CD, as described in International Patent Application Publication Nos. WO 2005/65435, WO 2005/065649, and WO 2005/065651, each of which are specifically incorporated by reference herein), hydroxyethyl, hydroxypropyl (including 2- and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β- and γ-cyclodextriiu, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999) and New Trends in Cyclodextrins and Derivatives (Ed. Dominique Duchene, Editions de Sante, Paris, France, 1991).

Suitable surface modifiers for use in the dosage formulation of the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the dosage formulation of the present invention include, but are not limited to, hydroxypropyl methylcellulose, hydroxypropylcellulose, polyvinylpyrrolidone, sodium lauryl sulfate, dioctylsulfosuccinate, gelatin, case in, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, e.g., Tween 20™ and Tween 80™ (ICI Specialty Chemicals)), polyethylene glycols (e.g., Carbowax 3550™ and 934™ (Dow Chemical)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68™ and F108™, which are block copolymers of ethylene oxide and propylene oxide), poloxamines (e.g., Tetronic 908™, also known as Poloxamine 908™, which is a tetrafanctional block copolymer derived from sequential addition of propylene oxide and ethylene oxide to ethylenediamine (BASF Wyandotte Corporation, Parsippany, N.J.)), Tetronic 1508™ (T-1508) (BASF Wyandotte Corporation), Tritons X-200™, which is an alkyl aryl polyether sulfonate (Rohm and Haas), Crodestas F-100™, which is a mixture of sucrose stearate and sucrose distearate (Croda Inc.), p-isononylphenoxypoly-(glycidol), also known as Olin-10G™ or Surfactant 10™ (Olin Chemicals, Stamford, Conn.), Crodestas SL-40.R™. (Croda, Inc.), and SA9OHCO, which is C18H37CH2(-CON(CH3)—CH2(CHOH)4(CH2OH)2 (Eastman Kodak Co.), decanoyl-N-methylglucamide, n-decyl-β-D-glucopyranoside, n-decyl-β-D-maltopyranoside, n-dodecyl β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucaamide, n-noyl-β-D-glucopyranoside, octanoyl-N-methylglucamide, n-octyl-β-D-glucopyranoside, octyl β-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like. (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Cationic stabilizers useful in the present dosage formulations, include, but are not limited to, cationic lipids, sulfonium, phosphonium, and quartemary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15 dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)4 ammonium chloride or bromide, N-alkyl (C12-18) dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, Mirapol™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides, imide azolinium salts, protonated quaternary acrylamides, methylated quaternary polymers, such as poly[diallyl dimethylammonium chloride] and poly-[N-methyl vinyl pyridinium chloride], and cationic guar.

In addition to dosage formulations comprising a β₂ agonist and a solubility enhancer, it is contemplated herein that dosage formulations formulated by methods which provide enhanced solubility are likewise suitable for use in the presently disclosed invention. Thus, in the context of the present invention, a “solubility enhancer” includes dosage formulations formulated by methods which provide enhanced solubility with or without a chemical agent acting as a solubility enhancer. Such methods include, e.g., the preparation of supercritical fluids. In accordance with such methods, β₂ agonist compositions, such as albuterol, are fabricated into particles with narrow particle size distribution (usually less than 200 nanometers spread) with a mean particle hydrodynamic radius in the range of 50 nanometers to 700 nanometers. The nano-sized β₂ agonist particles, such as albuterol particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Furthermore, the processes for producing nanometer sized particles, including SCF, can permit selection of a desired morphology (e.g., amorphous, crystalline, resolved racemic) by appropriate adjustment of the conditions for particle formation during precipitation or condensation. As a consequence of selection of the desired particle form, extended release of the selected medicament can be achieved. These particle fabrication processes are used to obtain nanoparticulates that have high purity, low surface imperfections, low surface charges and low sedimentation rates. Such particle features inhibit particle cohesion, agglomeration and also prevent settling in liquid dispersions. Additionally, because processes such as SCF can separate isomers of certain medicaments, such separation could contribute to the medicament's enhanced activity, effectiveness as well as extreme dose reduction. In some instances, isomer separation also contributes to reduced side effects. In accordance with the present methods and systems, an inhalation mixture can be a composition fabricated into a powdered form by any process including SCF, spray drying, precipitation and volume exclusion, directly into a collection media, wherein the particulate compound is thus automatically generated into a dispersed formulation. In some embodiments, this formulation can be the final formulation.

In certain embodiments, the dosage formulations of the present invention can further comprise a second pharmaceutically active agent. In certain embodiments, the second pharmaceutically active agent can be selected from (a) a corticosteroid; (b) an antibiotic; (c) an anti-cholinergic agent; or (d) a dopamine (D₂) receptor agonist.

Corticosteriods for use in the dosage formulations described herein include, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In one embodiment, the second pharmaceutically active agent is budesonide.

Antibiotics for use in the dosage formulations described herein include, but are not limited to, penicillins, cephalosporins, macrolides, sulfonamides, aminoglycosides, and β-lactam antibiotics.

Anticholinergic agents for use in the dosage formulations described herein include, but are not limited to, ipratropium bromide, oxitropium bromide, atropine methyl nitrate, atropine sulfate, ipratropium, belladonna extract, scopolamine, scopolamine methobromide, homatropine methobromide, hyoscyamine, isopriopramide, orphenadrine, tiotropium bromide and glycopyrronium bromide.

Dopamine (D₂) receptor agonists for use in the dosage formulations described herein include, but are not limited to, Apomorphine ((r)-5,6,6a,7-tetrahydro-6-methyl-4H-dibenzo[de,g]quinoline-10,11-diol); Bromocriptine ((5′α)-2-bromo-12′-hydroxy-2′-(1-methylethyl)-5′-(2-methylpropyl)erg otaman-3′,6′,18-trione); Cabergoline ((8β)-N-(3(dimethylamino)propyl)-N-((ethylamino)carbonyl)6-(2-propeny 1)ergoline-8-carboxamide); Lisuride (N′-((8α)-9,10-didehydro-6-methylergolin-8-yl)-N,N-diethylurea); Pergolide ((8β)-8-((methylthio)methyl)-6-propylergoline); Levodopa (3-hydroxy-L-tryrosine); Pramipexole ((s)-4,5,6,7-tetrahydro-N₆-propyl-2,6-benzothiazolediamine); Quinpirole hydrochirodie (trans-(−)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine). Other dopamine D₂ receptor agonists for use herein are disclosed in International Patent Application Publication No. WO 99/36095.

Other active ingredients for use in the inhalable compositions described herein include, but are not limited to, IL-5 inhibitors such as those disclosed in U.S. Pat. No. 5,668,110, No. 5,683,983, No. 5,677,280, No. 6,071,910 and No. 5,654,276, each of which is incorporated by reference herein; anti-sense modulators of IL-5 such as those disclosed in U.S. Pat. No. 6,136,603, the relevant disclosure of which is hereby incorporated by reference; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623, which is incorporated by reference herein; tachykinin receptor antagonists such as those disclosed in U.S. Pat. No. 5,691,336, No. 5,877,191, No. 5,929,094, No. 5,750,549 and No. 5,780,467, each of which is incorporated by reference herein; leukotriene receptor antagonists such as montelukast sodium (Singular, R-(E)]-1-[[[1-[3-[2-(7-chloro-2-quinolinyl)-ethenyl]-phenyl]-3-[2-(I-hydroxy-1-methylethyl)-phenyl]-propyl]-thio]-methyl] cyclopro-paneacetic acid, monosodium salt), 5-lypoxygenase inhibitors such as zileuton (Zyflo®, Abbott Laboratories, Abbott Park, Ill.), and anti-IgE antibodies such as Xolair (recombinant humanized anti-IgE monoclonal antibody (CGP 51901; IGE 025A; rhuMAb-E25), Genentech, Inc., South San Francisco, Calif.), and topical anesthetics such as lidocaine, N-arylamide, aminoalkylbenzoate, prilocaine, etidocaine (U.S. Pat. No. 5,510,339, No. 5,631,267, and No. 5,837,713, the relevant disclosures of which are hereby incorporated by reference).

In certain embodiments, the dosage formulations described herein can be used in the treatment of a patient diagnosed with, or suspected of having, a disease selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

III. Nebulizers for Use in the Present Methods

The methods described herein are intended for the delivery of a β₂ agonist to treat a disease by inducing bronchodilation in a patient in need thereof by an inhalation nebulizer. Suitable inhalation nebulizers include, e.g., jet nebulizers, ultrasonic nebulizers, pulsating nebulizers, and nebulizers comprising a vibrating mesh or plate with an aqueous chamber (e.g., Pari eFlow®, TouchSpray®, AeroNeb® Aerodose Inhaler, or Omron® NE-U03 NE-U22). In some embodiments, the nebulizers are available from, e.g., Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others. In certain embodiments, the nebulizer comprises a vibrating mesh or plate with an aqueous chamber. In one embodiment, the nebulizer is a Pari eFlow® nebulizer.

Other nebulizers suitable for use in the methods and systems described herein include, but are not limited to, jet nebulizers (optionally sold with compressors), ultrasonic nebulizers, and others. Exemplary jet nebulizers for use herein include Pari LC plus/ProNeb, Pari LC plus/ProNeb Turbo, Pari LCPlus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traveler, DeVilbiss 646, Whisper Jet, AcornII, Misty-Neb, Allied aerosol, Schuco Home Care, Lexan Plasic Pocet Neb, SideStream Hand Held Neb, Mobil Mist, Up-Draft, Up-DraftII, T Up-Draft, ISO-NEB, Ava-Neb, Micro Mist, and PulmoMate.

Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist Hand Held Ultrasonic Nebulizer. Other nebulizers for use herein include 5000 Electromagnetic Neb, 5001 Electromagnetic Neb 5002 Rotary Piston Neb, Lumnineb I Piston Nebulizer 5500, Aeroneb Portable Nebulizer System, Aerodose Inhaler, and AeroEclipse Breath Actuated Nebulizer.

In certain embodiments, nebulizers suitable for use in the presently described invention include nebulizers comprising a vibrating mesh or plate with an aqueous chamber. Such nebulizers are sold commercially as, e.g., Pari eFlow®, and are described in U.S. Pat. Nos. 6,962,151, 5,518,179, 5,261,601, and 5,152,456, each of which is specifically incorporated by reference herein. In still other embodiments, suitable nebulizers for use in the presently described include nebulizers comprising a vibrating mesh or plate with multiple apertures as described by R. Dhand in New Nebuliser Technology—Aerosol Generation by Using a Vibrating Mesh or Plate with Multiple Apertures, Long-Term Healthcare Strategies 2003, (July 2003), and p. 1-4 and Respiratory Care, 47: 1406-1416 (2002), disclosure of each of which is hereby incorporated by reference.

The parameters used in nebulization, such as flow rate, mesh membrane size, aerosol inhalation chamber size, mask size and materials, valves, and power source may be varied in accordance with the principles of the present invention to maximize their use with different types of inhalation mixtures or different types of β₂ agonists and delivery time conditions specified herein.

### EXAMPLES

The following ingredients, processes and procedures for practicing the methods described herein correspond to that described above. Any methods or materials not particularly described in the following examples are within the scope of the invention and will be apparent to those skilled in the art with reference to the disclosure herein.

### Example1

A patient experiencing asthma initiates treatment for asthma by inducing bronchodilation. The patient induces bronchodilation by placing a dose of an inhalation mixture comprising about 0.20 mg/dose of albuterol into the reservoir of a commercially available Pari eFlow® vibrating membrane inhalation nebulizer. The delivery of the inhalation mixture by the nebulizer is then initiated. Over the course of less than about three (3) minutes, the inhalation mixture is delivered with the inhalation nebulizer and the symptoms of asthma are ameliorated or relieved.

### Example 2

The same procedure is followed as in Example 1; however, in this case the symptoms of the patient are not sufficiently ameliorated after the initial dose is delivered over the course of less than about three (3) minutes. In response to the continued presence of the symptoms, the patient repeats the procedure as set forth in Example 1. Upon the completion of the second delivery of the inhalation mixture of albuterol, the symptoms of asthma are ameliorated or relieved.

### Example 3

Preparation and use of an inhalation mixture comprising a β₂ agonist, a solubility enhancer and a corticosteroid.

A patient experiencing asthma initiates treatment for asthma by inducing bronchodilation. The patient induces bronchodilation by: (i) adding a dose of an inhalation mixture comprising about 0.18 mg of free base albuterol and 5% (w/v) Captisol® (Cydex, Inc. Lenexa, Kans.) into the reservoir of a commercially available Pari eFlow® vibrating membrane inhalation nebulizer; (ii) adding a single Pulmicort® Respules (0.25 mg/2 ml) unit dose (AstraZeneca) to the albuterol inhalation mixture in the reservoir of the Pari eFlow® vibrating membrane inhalation nebulizer. The delivery of the inhalation mixture by the nebulizer is then initiated. Over the course of less than about 1.5 minutes, the inhalation mixture comprising (i) and (ii) is simultaneously delivered to the patient with the Pari eFlow® nebulizer and the symptoms of asthma are ameliorated or relieved.

### Example 4

A patient experiencing chronic obstructive pulmonary disease (COPD) initiates treatment by inducing bronchodilation. The patient induces bronchodilation by placing a dose of an inhalation mixture comprising about 0.18 mg/dose of formoterol into the reservoir of a commercially available Pari eFlow® vibrating membrane inhalation nebulizer. The delivery of the inhalation mixture by the nebulizer is then initiated. Over the course of less than about two (2) minutes, the inhalation mixture is delivered with the inhalation nebulizer and the symptoms of COPD are ameliorated or relieved.

### Example 5

A patient experiencing chronic obstructive pulmonary disease (COPD) initiates treatment by inducing bronchodilation. The patient induces bronchodilation by placing a dose of an inhalation mixture comprising about 0.20 mg/dose of albuterol sulfate into the reservoir of a commercially available Pari eFlow® vibrating membrane inhalation nebulizer. Over the course of less than about two (2) minutes, the inhalation mixture is delivered with the inhalation nebulizer and the symptoms of COPD are ameliorated or relieved.

### Example 6

Albuterol inhalation solutions were prepared from albuterol free base with varying concentrations according to the following specifications:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Hydrochloric acid (1 N) | ad ˜pH 3.75 ± 0.15 | ad ˜pH 3.75 ± 0.15 | ad ˜pH 3.75 ± 0.15 |
| Sodium Hydroxide (NaOH) | ad ˜pH 3.75 ± 0.15 | ad ˜pH 3.75 ± 0.15 | ad ˜pH 3.75 ± 0.15 |
| (optional) | | | |
| NaCl | ad ˜0.290 Osmol/kg | ad ˜0.290 Osmol/kg | ad ˜0.290 Osmol/kg |
| Water (weight %) | qs to 100 | qs to 100 | qs to 100 |

Three albuterol inhalation solutions (LOW, MEDIUM, and HIGH) having a total volume of approximately 100 mls were prepared using the following pharmaceutically acceptable agents:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Hydrochloric acid (1 N) (weight %) | 0.17 | 0.55 | 1.06 |
| NaCl (weight %) | 0.90 | 0.87 | 0.84 |
| Water (weight %) | qs to 100 | qs to 100 | qs to 100 |

The LOW concentration albuterol inhalation solution was formulated according to the following methods: (a) 902 mg NaCl was added to approximately 80 g water to form an aqueous solution; (b) 30 mg Albuterol Free Base was added to the solution of (a); (c) 170 mg HCl-1N was added to the solution of (b); and (d) approximately 20 g water was added to the solution of (c). The albuterol inhalation solution prepared according to this method has a pH=3.73 and an osmolality=0.288 Osmol/kg. The molar ratio of starting albuterol free base: hydrochloric acid was 0.871.

The MEDIUM concentration albuterol inhalation solution was formulated according to the following methods: (a) 875 mg NaCl was added to approximately 80 g water to form an aqueous solution; (b) 60 mg Albuterol Free Base was added to the solution of (a); (c) 550 mg HCl-1N was added to the solution of (b); and (d) approximately 20 g water was added to the solution of (c). The albuterol inhalation solution prepared according to this method has a pH=3.60 and an osmolality=0.286 Osmol/kg. The molar ratio of starting albuterol free base: hydrochloric acid was 1.076.

The HIGH concentration albuterol inhalation solution was formulated according to the following methods: (a) 840 mg NaCl was added to approximately 80 g water to form an aqueous solution; (b) 250 mg Albuterol Free Base was added to the solution of (a); (c) 1060 mg HCL-1N was added to the solution of (b); and (d) approximately 20 g water was added to the solution of (c). The albuterol inhalation solution prepared according to this method has a pH=3.41 and an osmolality=0.282 Osmol/kg. The molar ratio of starting albuterol free base: hydrochloric acid was 1.164.

The three concentrations of the albuterol inhalation solutions are filter sterilized and packaged into 0.5 ml unit doses by adding about 0.5 ml of the albuterol solutions into polyethylene unit dose vials. The resulting unit dose formulations will have a concentration of approximately 0.15 mg starting albuterol free base/dose, approximately 0.60 mg starting albuterol free base/dose, and approximately 1.25 mg starting albuterol free base/dose, respectively.

### Example 7

Albuterol inhalation solutions are prepared from albuterol free base with varying concentrations according to the following specifications:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Citric acid (anhydrous) | ad ˜pH 3.75 ± 0.15 | ad ˜pH 3.75 ± 0.15 | ad ˜pH 3.75 ± 0.15 |
| Sodium Citrate (optional) | ad ˜pH 3.75 ± 0.15 | ad ˜pH 3.75 ± 0.15 | ad ˜pH 3.75 ± 0.15 |
| NaCl | ad ˜0.290 Osmol/kg | ad ˜0.290 Osmol/kg | ad ˜0.290 Osmol/kg |
| Water (weight %) | qs to 100 | qs to 100 | qs to 100 |

Three albuterol inhalation solutions (LOW, MEDIUM, and HIGH) having a total volume of approximately 100 mls are prepared using the following pharmaceutically acceptable agents:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Citric acid (anhydrous) (weight %) | 0.03 | 0.11 | 0.21 |
| NaCl (weight %) | 0.90 | 0.87 | 0.84 |
| Water (weight %) | qs to 100 | qs to 100 | qs to 100 |

The LOW concentration albuterol inhalation solution was formulated according to the following methods: (a) 900 mg NaCl was added to approximately 80 g water to form an aqueous solution; (b) 30 mg Albuterol Free Base was added to the solution of (a); (c) 30 mg citric acid was added to the solution of (b); and (d) approximately 20 g water was added to the solution of (c). The albuterol inhalation solution prepared according to this method has a pH=3.76 and an osmolality=0.286 Osmol/kg. The molar ratio of starting albuterol free base: citric acid was 0.803.

The MEDIUM concentration albuterol inhalation solution was formulated according to the following methods: (a) 871 mg NaCl was added to approximately 80 g water to form an aqueous solution; (b) 60 mg Albuterol Free Base was added to the solution of (a); (c) 110 mg citric acid was added to the solution of (b); and (d) approximately 20 g water was added to the solution of (c). The albuterol inhalation solution prepared according to this method has a pH=3.76 and an osmolality-0.286 Osmol/kg. The molar ratio of starting albuterol free base: citric acid was 0.876.

The HIGH concentration albuterol inhalation solution was formulated according to the following methods: (a) 840 mg NaCl was added to approximately 80 g water to form an aqueous solution; (b) 250 mg Albuterol Free Base was added to the solution of (a); (c) 210 mg citric acid was added to the solution of (b); and (d) approximately 20 g water was added to the solution of (c). The albuterol inhalation solution prepared according to this method has a pH=3.78 and an osmolality=0.286 Osmol/kg. The molar ratio of starting albuterol free base: citric acid was 0.956.

The three concentrations of the albuterol inhalation solutions are filter sterilized and packaged into 0.5 ml unit doses by adding about 0.5 ml of the albuterol solutions into polyethylene unit dose vials. The resulting unit dose formulations will have a concentration of approximately 0.15 mg starting albuterol free base/dose, approximately 0.60 mg starting albuterol free base/dose, and approximately 1.25 mg starting albuterol free base/dose, respectively.

### Example 8

Albuterol inhalation solutions are prepared from albuterol free base with varying concentrations according to the following specifications:

| | Albuterol Concentration [mg/dose] | | |
|---|---|---|---|
| | 0.15 | 0.60 | 1.25 |
| Ingredient | LOW | MEDIUM | HIGH |
| Albuterol Free base (weight %) | 0.03 | 0.12 | 0.25 |
| H₃PO₄ (dilute) | ad ˜pH 3.75 ± 0.15 | ad ˜pH 3.75 ± 0.15 | ad ˜pH 3.75 ± 0.15 |
| Sodium Phosphate dibasic (optional) | ad ˜pH 3.75 ± 0.15 | ad ˜pH 3.75 ± 0.15 | ad ˜pH 3.75 ± 0.15 |
| NaCl | ad ˜0.290 Osmol/kg | ad ˜0.290 Osmol/kg | ad ˜0.290 Osmol/kg |
| Water (weight %) | qs to 100 | qs to 100 | qs to 100 |

Three albuterol inhalation solutions (LOW, MEDIUM, and HIGH) having a total volume of approximately 100 mls are prepared using the following pharmaceutically acceptable agents:

| | Albuterol Concentration [mg/dose] | | |
|---|---|---|---|
| | 0.15 | 0.60 | 1.25 |
| Ingredient | LOW | MEDIUM | HIGH |
| Albuterol Free base (weight %) | 0.03 | 0.12 | 0.25 |
| H₃PO₄ (dilute) (weight %) | 0.17 | 0.55 | 1.06 |
| NaCl (weight %) | 0.90 | 0.87 | 0.84 |
| Water (weight %) | qs to 100 | qs to 100 | qs to 100 |

The LOW concentration albuterol inhalation solution was formulated according to the following methods: (a) 901 mg NaCl was added to approximately 80 g water to form an aqueous solution; (b) 30 mg Albuterol Free Base was added to the solution of (a); (c) 100 mg H₃PO₄ 30 mg was added to the solution of (b); and (d) approximately 20 g water was added to the solution of (c). The albuterol inhalation solution prepared according to this method has a pH=3.72, and an osmolality=0.288 Osmol/kg.

The MEDIUM concentration albuterol inhalation solution was formulated according to the following methods: (a) 873 mg NaCl was added to approximately 80 g water to form an aqueous solution; (b) 60 mg Albuterol Free Base was added to the solution of (a); (c) 340 mg H₃PO₄ was added to the solution of (b); and (d) approximately 20 g water was added to the solution of (c). The albuterol inhalation solution prepared according to this method has a pH=3.77 and an osmolality=0.285 Osmol/kg.

The HIGH concentration albuterol inhalation solution was formulated according to the following methods: (a) 840 mg NaCl was added to approximately 80 g water to form an aqueous solution; (b) 250 mg Albuterol Free Base was added to the solution of (a); (c) 1020 mg H₃PO₄ was added to the solution of (b); and (d) approximately 20 g water was added to the solution of (c). The albuterol inhalation solution prepared according to this method has a pH=3.67 and an osmolality=0.283 Osmol/kg.

The three concentrations of the albuterol inhalation solutions are filter sterilized and packaged into 0.5 ml unit doses by adding about 0.5 ml of the albuterol solutions into polyethylene unit dose vials. The resulting unit dose formulations will have a concentration of approximately 0.15 mg starting albuterol free base/dose, approximately 0.60 mg starting albuterol free base/dose, and approximately 1.25 mg starting albuterol free base/dose, respectively.

### Example 9

A patient experiencing asthma initiates treatment for asthma by inducing bronchodilation. The patient induces bronchodilation by placing a single unit dose of an inhalation mixture comprising about 1.25 mg/dose of albuterol as described in Example 6 into the reservoir of a commercially available Pari eFlow® vibrating membrane inhalation nebulizer. The delivery of the inhalation mixture by the nebulizer is then initiated. Over the course of less than about three (3) minutes, the inhalation mixture is delivered with the inhalation nebulizer and the symptoms of asthma are ameliorated or relieved.

### Example 10

Three albuterol inhalation solutions are prepared according to the methods set forth below using the following pharmaceutically acceptable agents:

| | Albuterol Concentration [mg/dose] | | |
|---|---|---|---|
| | 0.31 | 0.63 | 1.25 |
| Ingredient | LOW | MEDIUM | HIGH |
| Albuterol free base (weight g) | 0.062 | 0.126 | 0.25 |
| Sodium Chloride NaCl (weight g) | 0.88 g | 0.86 g | 0.84 g |
| Hydrochloric acid (HCl) (1 N) | ad ˜pH 3.75 ± 0.15 | ad ˜pH 3.75 ± 0.15 | ad ˜pH 3.75 ± 0.15 |
| Sodium Hydroxide (NaOH) (1 N) | ad ˜pH 3.75 ± 0.15 | ad ˜pH 3.75 ± 0.15 | ad ˜pH 3.75 ± 0.15 |
| Water (weight mL) | qs to 100 | qs to 100 | qs to 100 |

The albuterol inhalation solutions are formulated according to the following methods: (a) NaCl is added to approximately 80 mL water to form an aqueous solution; (b) albuterol free base is added to the solution of (a); (c) HCl is added to the solution of (b); and (d) approximately 20 mL water is added to the solution of (c). The pH is determined using standard chemical techniques known in the art. If the pH of the aqueous inhalation solution comprising (a)-(d) is lower than 3.75±0.15, the process further comprises (e) the addition of NaOH until a pH of 3.75±0.15 is obtained

### Example 11

Clinical evaluation was conducted by performing gamma scintigraph analysis on subjects after administration via nebulization of the albuterol inhalation solutions as described herein. The purpose of the study was to compare, by gamma scintigraphy, the intra-pulmonary deposition of radio-labeled albuterol following nebulization of the albuterol inhalation solutions by a Pari eFlow device against the intra-pulmonary deposition of radio-labeled albuterol following nebulization of Ventolin Nebules® (Allen & Hanburys, UK, 2.5 mg albuterol/2.5 mL).

Each study subject receive each study treatment (radiolabelled with Technetium-99m (99mTc) chelated with diethylene triamine penta-acetic acid (DTPA)) as a single dose treatment over three treatment periods in a cross-over design and with a minimum of 3 days and a maximum of 14 days of wash-out period between each Treatment Visit. The albuterol inhalation solution treatments were administered via pulmonary inhalation using eFlow nebuliser (PARI, Germany) whereas LC Plus nebuliser (PARI, Germany) was used to administer the Ventolin Nebules®.

J Two doses of the albuterol inhalation solution were tested in this study: (a) 0.63 mg salbutamol in 0.5 mL solution, and (b) 1.25 mg salbutamol in 0.5 mL solution. The albuterol inhalation solutions were prepared by the methods described in Example 10 and contain albuterol (active), Sodium Chloride (tonicity adjustment), and Hydrochloric acid and Sodium Hydroxide (pH adjustment). These dosage forms were administered by a Pari eFlow nebulizer for a period of time ranging from 1 minute to 3 minutes. One dose of (c) Ventolin Nebules® (Allen & Hanburys, UK, 2.5 mg/2.5 mL) was tested this study. The Ventolin® solution was further diluted to 3 mL using saline as the diluting fluid. This dosage form was administered by a Pari LC Plus nebulizer for period of time until the nebulizer began sputtering (time to sputter (TTS)), which was between 5 and 10 minutes.

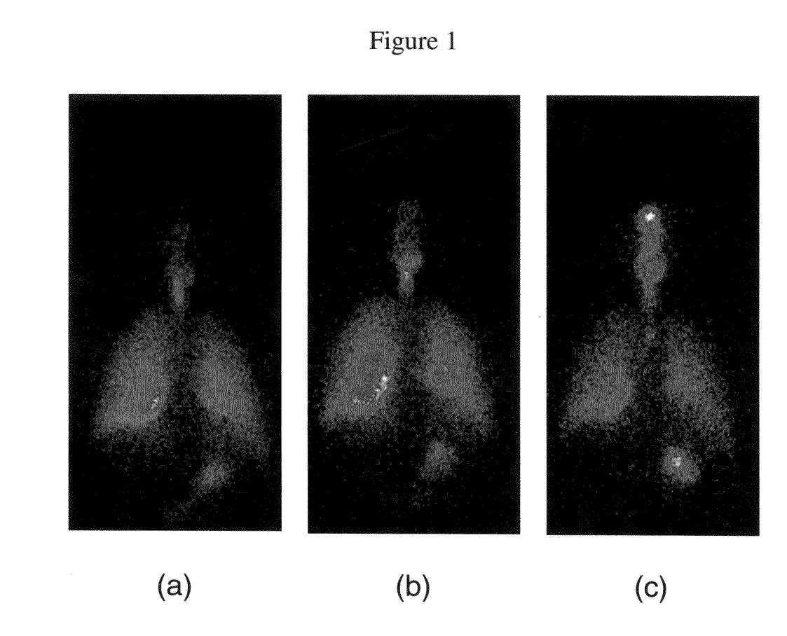

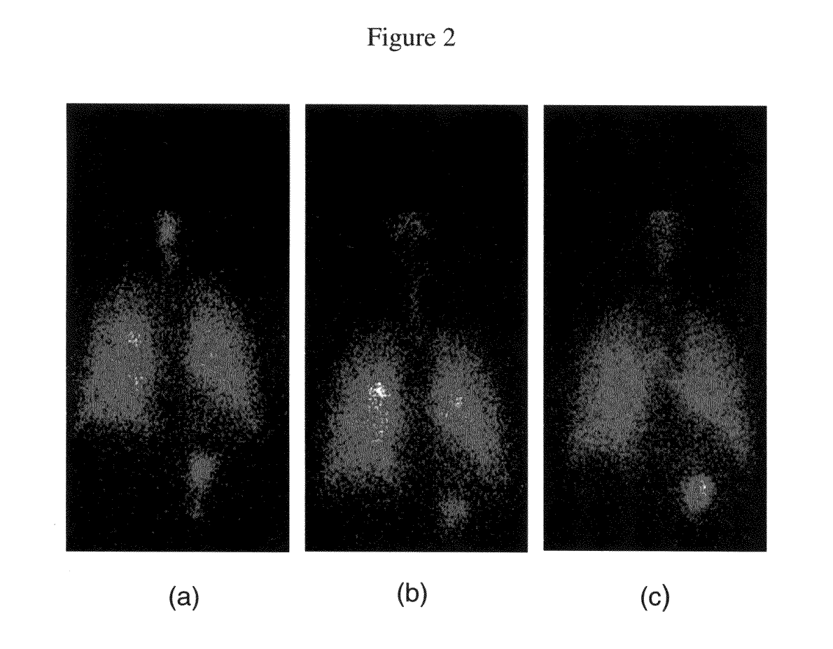

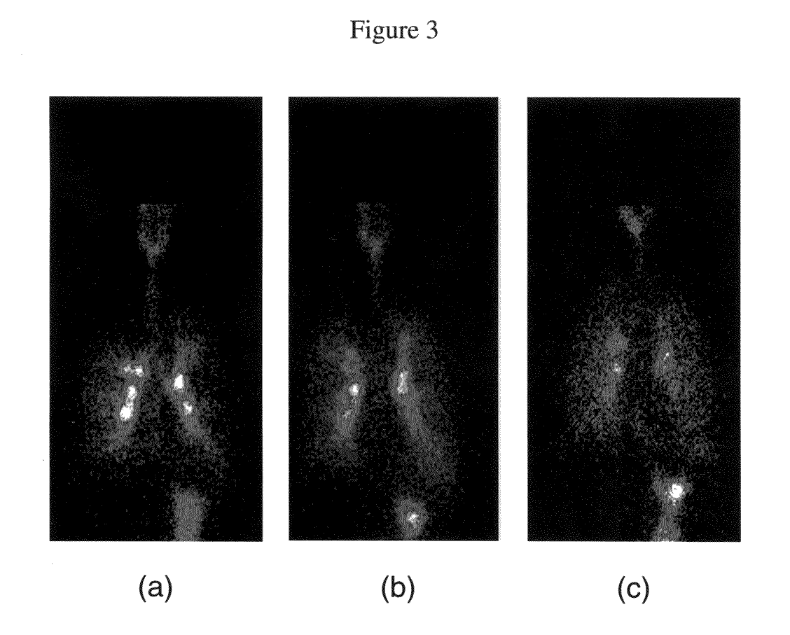

FIGS. 1-3 set forth the scintigraphic data for the above study. As indicated in FIGS. 1-3, the administration of the albuterol inhalation solution of the present invention with an Pari eFlow nebulizer resulted in increased lung deposition as compared to administration of the Ventolin® solution with a Pari LC Plus nebulizer.

1. A method for treating a disease by inducing bronchodilation in a patient in need thereof, said method comprising:
(a) providing at least one dose of an inhalation mixture comprising a β₂ agonist said patient; and
(b) delivering the inhalation mixture with an inhalation nebulizer for less than about 5 minutes,
wherein at least about 20% of the β₂ agonist is deposited in the lung.

2. A method for treating a disease by inducing bronchodilation in a patient in need thereof, the method comprising:
(a) providing at least one dose of an inhalation mixture comprising a β₂ agonist to said patient; and
(b) delivering the inhalation mixture with an inhalation nebulizer for less than about 5 minutes,
wherein less than about 30% of the β₂ agonist is delivered outside of the lung.

3. (canceled)

4. A method for treating a disease by inducing bronchodilation in a patient in need thereof, said method comprising:
(a) providing at least one dose of an inhalation mixture comprising a β₂ agonist; and
(b) delivering the inhalation mixture with an inhalation nebulizer for less than about 5 minutes,
whereby said method provides equivalent bronchodilation in said patient as compared to traditional β₂ agonist treatments at a β₂ agonist dose lower than traditional β₂ agonist treatments.

5. A method for treating a disease by inducing bronchodilation in a patient in need thereof, said method comprising:
(a) providing at least one dose of an inhalation mixture comprising a β₂ agonist; and
(b) delivering the inhalation mixture with an inhalation nebulizer for less than about 5 minutes,
wherein said method provides greater bronchodialation in said patient as compared to traditional β₂ agonist treatments of the same β₂ agonist dose.

6. The method of claim 1, wherein said β₂ agonist is a short acting β₂ agonist selected from the group consisting of albuterol, albuterol free base, albuterol sulfate, albuterol hydrochloride, albuterol maleate, albuterol tartrate, albuterol citrate, albuterol phosphate, terbutaline sulfate, bitolterol mesylate, levalbuterol, metaproterenol sulfate, pirbuterol acetate, and combinations thereof.

7-14. (canceled)

15. The method of claim 1, further comprising delivering a second pharmaceutically active agent in an inhalation mixture with an inhalation nebulizer.

16. The method of claim 15, wherein said second pharmaceutically active agent is a selected from the group consisting of a corticosteroid, an antibiotic, an anti-cholinergic agent, or a dopamine (D₂) receptor agonist.

17-21. (canceled)

22. The method of claim 15, wherein said inhalation mixture comprising a β₂ agonist and said inhalation mixture comprising a second pharmaceutically active agent are delivered simultaneously.

23. The method of claim 15, wherein said inhalation mixture comprising a β₂ agonist and said inhalation mixture comprising a second pharmaceutically active agent are delivered consecutively.

24. The method of claim 1, wherein said patient is an adult over 18 years of age.

25. The method of claim 1, wherein said patient is an adolescent between the ages of 12 and 18 years of age.

26-27. (canceled)

28. The method of claim 1, wherein said patient is a child between the ages of 2 and 12 years of age.

29. (canceled)

30. The method of claim 1, wherein said patient is an infant less than 2 years of age.

31. The method of any of claim 1, wherein said delivering with said inhalation nebulizer is for less than about 4, about 3, about 2, or about 1.5, or about 1 minutes, or between about 30 seconds and 1 minutes, between about 1 and 2 minutes, between about 1 and 3 minutes, between about 2 and 3 minutes, or between about 3 and 4 minutes.

32. The method of claim 1, wherein the volume of said dose or inhalation mixture comprising a β₂ agonist is from about 0.1 ml to about 1.5 ml, from about 0.1 ml to about 1.0 ml, from 0.3 ml to about 0.8 ml or from about 0.4 ml to about 0.6 ml.

33-36. (canceled)

37. The method of claim 1, wherein at least about 25%, at least 30%, at least about 40%, at least about 50%, at least about 60%, at least about 70%, or at least about 80%, or between 30% and 40% of the β₂ agonist is deposited in the lung.

38. The method of claim 2, wherein less than about 25%, less than about 20%, less than about 15%, less than about 10% or less than about 5% of the β₂ agonist is delivered outside the lung.

39. (canceled)

40. The method of claim 1, wherein said nebulizer is a Pari eFlow nebulizer.

41. method of claim 1, wherein said method is a treatment for, or said patient is diagnosed with, or suspected of having, a disease selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

42. A dosage formulation for administration by inhalation nebulization comprising:
(a) a β₂ agonist or a pharmaceutically acceptable salt thereof;
(b) a preservative;
whereby said formulation is suitable for delivery by an inhalation nebulizer and said delivery takes less than about 5 minutes.

43. The dosage formulation of claim 42, wherein said β₂ agonist is a short acting β₂ agonist selected from the group consisting of albuterol, albuterol free base, albuterol sulfate, albuterol hydrochloride, albuterol maleate, albuterol tartrate, albuterol citrate, albuterol phosphate, terbutaline sulfate, bitolterol mesylate, levalbuterol, metaproterenol sulfate, pirbuterol acetate, and combinations thereof.

44-50. (canceled)

51. The dosage formulation of claim 42, wherein said preservative is selected from the group consisting of edetate disodium (EDTA), benzalkonium chloride (BAC), and combinations thereof.

52. (canceled)

53. The dosage formulation of claim 42, further comprising a solubility enhancer.

54. (canceled)

55. The dosage formulation of claim 42, further comprising a pharmaceutically acceptable excipient selected from the group consisting of a chelating agent, a sequestering agent, or an antioxidant.

56. The dosage formulation of claim 42, further comprising a second pharmaceutically active agent selected from the group consisting of a corticosteroid, an antibiotic, an anti-cholinergic agent, or a dopamine (D₂) receptor agonist.

57-60. (canceled)
